(19) United States
(12) Patent Application Publication
(10) Pub. No.: US 2025/0160307 A1
(43) Pub. Date: May 22, 2025

(54) BRAIN TUMOR MODEL

(71) Applicant: Emory University, Atlanta, GA (US)

(72) Inventors: Nicholas M. Boulis, Atlanta, GA (US); Muhibulla S. Tora, Atlanta, GA (US); Thais Buchman, Atlanta, GA (US)

(21) Appl. No.: 18/955,495

(22) Filed: Nov. 21, 2024

Related U.S. Application Data
(60) Provisional application No. 63/601,406, filed on Nov. 21, 2023.

(51) Int. Cl. A01K 67/0276; C12N 5/079; C12N 15/113; C12N 15/86

(52) U.S. Cl. CPC A01K 67/0276; C12N 5/0618; C12N 15/1135; C12N 15/1136; C12N 15/1137; C12N 15/86; A01K 2207/05; A01K 2217/058; A01K 2227/108; A01K 2267/0331; C12N 2310/14; C12N 2310/531; C12N 2740/15043; C12Y 101/01042

(57) ABSTRACT

The present disclosure relates to methods for inducing intracranial tumor in a non-human animal. The disclosure also relates to an intracranial cancer engineered non-human animal model with a combination of viral vectors encoding oncogenes or shRNA targeting tumor suppressor genes and a population of intracranial cancer cells derived therefrom.

### CROSS-REFERENCE TO RELATED APPLICATIONS

This application claims the benefit of priority to U.S. Provisional Patent Application No. 63/601,406, filed on Nov. 21, 2023, the disclosure of which is expressly incorporated by reference herein in its entirety.

### STATEMENT REGARDING FEDERALLY SPONSORED RESEARCH

This invention was made with government support under grant RO1CA251393 awarded by the National Institutes of Health. The government has certain rights in the invention.

### FIELD

Disclosed herein are methods and a model of inducing intracranial tumors in an animal using a combination of viral vectors.

### BACKGROUND

Glioblastoma (GBM) is the most common and most deadly primary brain tumor in adults. This represents the recalcitrance of a disease process that is refractory to the standard of care and highlights the importance of development of topical animal models that can serve as pre-clinical neurosurgical platforms for the pre-clinical testing of neurosurgical therapies as well as other therapies that depend on an animal with an immune system and life expectancy mirroring that of humans more closely than rodents. Unfortunately, there have been limited improvements in outcomes. While there have been numerous surgical translational studies demonstrating anti-tumor activity in rodents, the anatomy of such models presents limitations that are difficult to scale into humans. These include, but are not limited to, decreased connective tissue, decreased white matter volume, thin meninges, more intimate dura mater, lower CSF volume, and lissencephalic brain structure. In terms of size alone, the murine brain is approximately 4 mm in diameter, as compared to the human brain at up to 140 mm. There is a need for an alternative, the large animal mammalian system that overcomes many of these anatomic limitations and has been increasingly employed in neuropathologic disease modeling as a putative preclinical platform. In addition to the absence of a reproducible large animal model, it follows that there is no large animal model for examination of explanted tumor samples to investigate the utility of allograft models and GBM organoids.

In human GBM, the RTK/RAS/PI3K pathway is widely implicated and affects up to 88% of cases. The second most commonly affected pathway is the p14Arf/CDKN2A and TP53 axis which is involved in at least 50% of cases. Targeting these pathways to drive gliomagenesis have been shown to induce GBM formation in rodent models. In mice, it is reported that additional genetic lesions may be required to model high-grade gliomagenesis with either constitutive RAS or knockdown of CDKN2A or p53.

Large animal models for translational oncologic research are an evolving field with numerous examples arising including severe combined immunodeficiency pigs (SCID) with transplantation of xenografts or modified stem cells, as well as genetically modified pigs with inducible expression of TP53R167 or KRASG12D (oncopig). The rational underlying this rise in popularity is principally due to two reasons for failure of new oncologic therapeutics in clinical trials, namely toxicity or lack of efficacy. This is especially true for neuro-oncologic tumors, particularly GBM, where few therapeutic strategies move beyond Phase I trials, and ultimately patients continue to face an abysmal prognosis. Currently, for modeling GBM, only two groups have used either U87 or G6 cell line based xenografts with concurrent immunosuppression, transplanting them into Landrace farm-pigs or Yucatan minipigs with macroscopic and histopathologic tumor growth. The use of G6 only produced tumor formation in only one of six animals. However, U87 cell line xenografts were more penetrant in tumor formation yielding tumor growth in 27 of 20 animals (93%) across three studies.

Despite significant advances in the knowledge of GBM, there have been limited improvements in outcomes. In part, this represents the recalcitrance of GBM. On the other hand, this highlights the limitations of surgical translation based on existing rodent models, which has stifled the translation of neurosurgical strategies. The study challenges the notion of pre-clinical translational reliance on phenotypically normal large mammalian models used by investigators and regulatory bodies in the pathway for developing neurosurgical strategies. This is a major limitation. For example, in prior studies to bring novel drug delivery strategies to clinical trials, groups have been unable to rigorously study drug delivery variables or drug tracking in a large animal disease model. As such, the development of a highly characterized large mammalian model of GBM fills a translational gap in the field for the study pre-clinical neurosurgical strategies. Indeed, the development of numerous technologies including convection enhanced delivery, oncolytic vectors, laser interstitial thermal therapy (LITT), focused ultrasound (FUS), intraoperative surgical guidance (e.g. 5-ALA), intra-arterial delivery, and robotic resection would benefit from a readily available highly characterized immunocompetent large animal model of GBM.

The absence of a reproducible, immunocompetent, and surgically relevant large animal glioma model that can be rapidly created from normal adult animals is a major gap in the field that stifled translational glioma research. Therefore, there is a need in the art for a large animal model to be employed in a variety of critical research applications. Most importantly, to pave the way for further advanced preclinical modeling of high-grade glioma as well as adaptation by preclinical neurosurgical and neuro-oncologic development programs.

Overall, there is a significant need for a novel strategy for assessing viral infection complications in brain cancer patients and thereby providing effective adaptive immune responses. Therefore, what is needed are new methods for diagnosing and treating cancer patients.

### SUMMARY

Disclosed herein are methods and models for inducing intracranial cancer in an animal for determining anticancer therapies.

In some examples, disclosed herein is a large non-human mammal model of intracranial cancer, comprising:
an intracranial cancer engineered large non-human mammal with a combination of viral vectors encoding oncogenes and shRNA targeting tumor suppressor genes; and
a population of intracranial cancer cells from the large non-human mammal model.

In some examples, the shRNA targeting tumor suppressor genes comprises sh787 or sh944, wherein the sh787 is operably linked to an H1 promoter, and sh944 is operably linked to a U6 promoter

In some examples, the tumor suppressor genes are CDKN2A, PTEN or p53.

In some examples, the oncogenes comprise platelet-derived growth factor receptor alpha (PDGFRA), platelet-derived growth factor beta (PDGFRB), mutant or wild-type isocitrate dehydrogenase (IDH), mutant or wildtype epidermal growth factor receptor (EGFR), or mutant histone H3.3 or mutated Harvey rat sarcoma viral oncogene (HRAS-G12V), wherein the PDGFRA, PDGFRB, IDH, EGFR, H3.3 or HRAS-G12V is operably linked to an Ef1α promoter.

In some examples, the viral vectors comprise a recombinant adenoviral vector, a recombinant adeno-associated viral vector (AAV), a herpes simplex virus type 1 vector (HSV), a moloney murine leukemia virus (MMLV) vector or a lentiviral vector.

In some examples, each viral vector in the combination is encapsulated in a nanoparticle, a polymer, or a liposome.

In some examples, the large non-human mammal model is a Gottingen minipig.

In some examples, the intracranial cancer comprises brain metastasis, glioblastoma, meningioma, cerebral arteriovenous malformation, vestibular schwannoma, pituitary adenoma, neuroblastoma, or gliosarcoma.

In some examples, the intracranial cancer is glioblastoma, wherein the glioblastoma comprises high-grade glioma (HGG) or low-grade glioma (LGG).

In some examples, disclosed herein is a method of growing an intracranial tumor in a large non-human mammal model, comprising:
administering a combination of viral vectors encoding oncogenes and shRNA targeting tumor suppressor genes at an intracranial delivery location; thereby inducing intracranial tumor.

In some examples, the method further comprises:
obtaining a biopsy sample from the intracranial tumor; and
culturing cells from the biopsy sample.

In some examples, the intracranial delivery location is determined using neuronavigation, wherein the neuronavigation comprises stereotactic targeting.

In some examples, the biopsy sample is obtained after 4 weeks of administering a combination of viral vectors in the large non-human mammal model.

In some examples, the combination of viral vectors comprises each viral vector titer at about 10⁸ to about 10¹⁰ infectious units (IU)/ml.

In some examples, the combination of viral vectors encoding oncogenes and shRNA targeting tumor suppressor genes are delivered concurrently or sequentially.

In some examples, the combination of viral vectors is administered by an intraparenchymal injection route.

In some examples, the combination of viral vectors is administered in an injection volume ranging from about 10 μL to about 50 μL.

In some examples, the intracranial delivery location is supratentorial, wherein the supratentorial location comprises subcortical white matter, deep white matter, or deep nuclear structures. In some examples, the intracranial delivery location is infratentorial, wherein the location compromises the brainstem or cerebellar white matter.

In some examples, disclosed herein is a method for screening an anticancer agent in a large non-human mammal model of intracranial cancer, comprising:
administering an anticancer agent to the large non-human mammal model;
wherein the large non-human mammal model comprises an intracranial tumor;
analyzing tumor suppression by the anticancer agent on the intracranial tumor; and
determining therapeutic efficacy of the anticancer agent.

In some examples, disclosed herein is a recombinant viral vector, comprising:
an epidermal growth factor receptor (EGFR) gene and a short hairpin RNA targeting phosphatase and tensin homolog gene (shPTEN), wherein the recombinant viral vector induces formation of infiltrating glioma in a large non-human mammal model.

In some examples, disclosed herein is a recombinant viral vector, comprising: a platelet-derived growth factor B (PDGFB) gene and a short hairpin RNA targeting the p53 gene (shP53), wherein the recombinant viral vector induces diffuse low-grade glioma in a large non-human mammal model.

In some examples, disclosed herein is a recombinant viral vector, comprising:
a BRAF V600E gene, a platelet-derived growth factor B (PDGFB) gene, and a short hairpin RNA targeting the p53 gene (shP53), wherein the recombinant viral vector induces sarcoma in a large non-human mammal model.

In some examples, disclosed herein is a recombinant viral vector, comprising:
a platelet-derived growth factor B (PDGFB) gene, a histone H3 lysine 27 to methionine mutant gene (H3K27M), and a short hairpin RNA targeting the CDKN2A gene (shCDKN2A), wherein the recombinant viral vector induces diffuse low-grade glioma in a large non-human mammal model.

In some examples, disclosed herein is a recombinant viral vector, comprising:
a PDGFB gene, an HRAS gene, and a shP53 gene, wherein the recombinant viral vector induces glioblastoma in a large non-human mammal model.

In some examples, disclosed herein is a combination of recombinant viral vectors to induce brain disorders in a large non-human mammal model, comprising:
a gene, wherein the gene consists of epidermal growth factor receptor (EGFR), platelet-derived growth factor B (PDGFB), Harvey rat sarcoma viral oncogene homolog (HRAS), epidermal growth factor receptor (EGFRvIII), B-Raf proto-oncogene, serine/threonine kinase mutation at valine 600 to glutamic acid (BRAF V600E), histone H3 mutation at lysine 27 to methionine (H3K27M), wild-type isocitrate dehydrogenase 1 (IDH1WT), isocitrate dehydrogenase 1 mutation at arginine 132 to histidine (IDH1 R132H), and isocitrate dehydrogenase 1 (IDH1); and
a short hairpin RNA targeting tumor suppressor genes, wherein the tumor suppressor genes consist of phosphatase and tensin homolog (PTEN), cyclin-dependent kinase inhibitor 2A (CDKN2A), and protein TP53 (p53).

### BRIEF DESCRIPTION OF THE DRAWINGS

The accompanying figures, which are incorporated in and constitute a part of this specification, illustrate several examples described below.

FIGS. 1A, 1B, 1C, 1D, 1E, 1F and 1G show experimental design and clinical findings. FIG. 1A shows lentiviral vectors expressing PDGFB, HRASG12V, and shRNA targeting P53 (sh787, sh944) designed based on prior literature at a titer of 3×10⁹ IU/ml. FIG. 1B shows vectors were injected into the subcortical white matter of mature minipigs. Animals were followed with clinical and behavioral evaluation until a pre-determined 4-week endpoint. Post-mortem neuropathologic evaluation was conducted to evaluate GBM growth. FIG. 1C shows two animals with 50 μL injections reached endpoint by post operative day 8 and 14. Two animals with 25 μL injections survived until the pre-determined 4-week endpoint. FIG. 1D shows baseline and endpoint weights were compared, demonstrating weight loss in the 50 μL group animals not the 25 L animals. FIG. 1E shows mental status declined to a PNS score of ⅓ among animals with 50 μL injections, but not 25 μL injections. Animals undergoing 50 μl injections experienced inappetence by endpoint, refusing solids and liquids (PNS appetite score=1), and refusing solids (PNS appetite score=2). Animals with 25 μL injections exhibited no changes in appetite.

FIGS. 2A, 2B and 2C show endpoint MRI and histopathologic features. FIG. 2A shows T1 weighted axial scans demonstrate a heterogeneous contrast-enhancing lesion with mass effect and effacement of the lateral ventricle (T1w+C, Sagittal, Axial, Coronal). T2 weighted FLAIR 3d maximal intensity projection demonstrates marked perilesional edema (T2w FLAIR MIP). Representative images from 25 μL injected animal at post-operative week 4. Comparative panels of axial scans at 1 cm depth from cortical surface. T1 weighted pre-contrast (T1w) demonstrates a region of hypo/iso intensity. T1w post-gadolinium scan (T1w+C) shows heterogenous enhancement. Gradient echo (GRE) scans display signal void and blood products and T2 fluid attenuated inversion recovery (T2-FLAIR) show peri-tumoral edema. FIG. 2B shows low magnification H & E demonstrates hypercellular region with hyperchromasia invading along white matter tracts, violation of grey and white matter borders, hemorrhage, and extension of the tumor to the cortical surface. Numerous regions of necrosis and thrombosed blood vessels are appreciated (20× magnification). Anaplastic astrocytic morphology is present with spindled and epithelioid cells (20× magnification). In addition, inflammatory regions are noted within the leptomeningeal space. FIG. 2C shows tumors show weak P53 and strong PDGFB and HRAS immunopositivity, as well as strong immunopositivity for redox markers C/EBPβ, NQO1, and GSTP1. Bar: 100 μm.

FIGS. 3A and 3B show tumor markers and microenvironmental features. FIG. 3A shows tumors demonstrate a high Ki-67 index, immunopositivity for glioma markers GFAP, Olig2, SOX2, PDGFRA, as well as Vimentin and NG2. Neurofilament staining is appreciated in the tumor illustrating presence of background parenchyma within the tumor bulk. Bar: 100 μm. FIG. 3B shows IF staining of endothelial cell/pericyte and basement membrane markers demonstrated significantly increased Desmin (P<0.01), CD31 (P<0.01), and Type IV Collagen (P<0.05) versus control. Bar: 50 μm.

FIGS. 4A, 4B and 4C show initial characterization of porcine glioma tissue culture. FIG. 4A shows tumor cell proliferation was significantly higher (P<0.05) than control cells at 6 days. FIG. 4B shows immunofluorescent staining of cells show expected positive vector reporters GFP and mCherry in both tumor and cultures. Bar: 20 μm. FIG. 4C shows immunofluorescent staining shows strong immunopositivity for GFAP, Olig2, PDGFRA, and CD68 compared to control. Bar: 50 μm.

FIGS. 5A, 5B, 5C and 5D show trajectory planning and surgical technique using the Medtronic StealthStation™ S8 surgical navigation system. FIG. 5A shows a planned trajectory targeting the thalamus with distance to target. FIG. 5B shows EM Tracer Pointer placed in the burr hole made at the entry point in the outer table of the skull. FIG. 5C shows EM stylet placed down the biopsy trajectory guide tube at the ultimate pre-determined depth set for injection. FIG. 5D shows a delivery system containing a 22-gauge 8 in. long blunt needle connected to a gastight Hamilton syringe placed down the guide tube in the biopsy trajectory guide tube and clamped at the ultimate pre-determined depth set for injection.

FIG. 6 shows injections into subcortical structures that produce bilateral highly penetrant lesions.

FIG. 7 shows unilateral lesion. (LEFT) T1 post weighted imaging, where a hypointense region can be appreciated; (RIGHT) gross necropsy shows correspondent significant hemorrhage at the ipsilateral injection site and target location.

FIG. 8 shows a 3-plane view (axial, sagittal, coronal) of an MRI scan overlaid on the CT pre-operative neuronavigation trajectory plan where the trajectory from the entry point (skin) to the target (cross hairs) is displayed (dotted line) with a total distance of 59 mm and enhancing tumor (arrow) can be seen.

FIGS. 9A and 9B show an immunohistochemistry for olig2 and Ki67 in spinal cord sections at 180 days post injection. (FIG. 9A) control, and (FIG. 9B) PDGFB+shP53 group. Low grade glioma (LGG) defined by cellular proliferation and nuclear atypia, without necrosis or vascular proliferation, created by injecting vectors for shP53 and PDGFB. Similar lesions were created with shPTEN/CDKN2A or EGFR.

FIGS. 10A, 10B and 10C show lentiviral vector-induced high-grade glioma growth in the minipig brain-refined methods (FIG. 10A) intracranial injection using stealth neuronavigation system for the planning of trajectories. (FIG. 10B and FIG. 10C) gross pathology and H and E preliminary stain showing masses in both hemispheres. H & E histology showing deep brain lesions with gross pathology sections and stereotactic injections with the Navigus tool guided by Axiom Stealth Navigation.

### DETAILED DESCRIPTION

Disclosed herein are methods and models for inducing intracranial cancer in a large non-human mammal model for determining anticancer therapies.

Intracranial or brain cancer, also known as a malignant brain tumor, is a fast-growing, life-threatening cancer that can invade and destroy brain tissue. Intracranial or brain cancer can be caused by tumors that start in the brain (primary brain tumors) or spread to the brain from other parts of the body (metastatic brain tumors). Intracranial cancers lead to headaches, seizures, difficulty in speaking, paralysis, balance problems or dizziness, vision issues, or hearing issues. Some exemplary intracranial cancers include but are not limited to brain metastasis, glioblastoma, meningioma, cerebral arteriovenous malformation, vestibular schwannoma, neuroblastoma, gliosarcoma, or pituitary adenoma. In some examples, the intracranial cancer is glioblastoma, wherein the glioblastoma comprises high-grade glioma (HGG) or low-grade glioma (LGG). Below is a table, depicting the differences between HGG and LGG. As used herein, a “glioma” refers to a tumor that forms in the brain or spinal cord from glial cells, which support and protect nerve cells. Gliomas are the most common primary brain tumors and can be benign or malignant. They can occur in adults and children, and are highly treatable and curable.

So far, glioma models have evolved to encompass a wide range of systems that replicate the biology and complexity of these tumors. Traditional in vitro models include glioma cell lines such as U87MG, U251, and T98G, alongside patient-derived cells and advanced 3D spheroids and organoids that better mimic tumor architecture and microenvironment. In vivo models, including genetically engineered mouse models (GEMMs), syngeneic systems, and patient-derived xenografts (PDX), provide insights into glioma development, progression, and therapeutic responses within a living organism. Large non-human mammals, such as pigs with their large, gyrencephalic brains, provide a promising model for studying glioma progression and testing technologies like intraoperative imaging systems. These large animal models bridge the gap between small animal research and human clinical trials, offering critical insights into glioma biology and therapeutic strategies.

As used herein, the model relies on the use of inbred strains, specifically the Gottingen Minipig, rather than outbred strains such as the Farm Pig. Experimental data demonstrates that while the HRAS/shP53/PDGFB combination failed to induce tumor formation in Farm Pigs, it consistently resulted in high-grade gliomas (HGG) in the brain and spinal cord of Gottingen Minipigs. Specific shRNA targeting tumor suppressors and oncogenes implicated in human gliomas have been shown to cause tumor formation, with the HGG combination previously disclosed for the spinal cord but not the brain. Additionally, shRNA targeting these tumor suppressors and oncogenes in the spinal cord, not previously disclosed, caused low-grade gliomas (LGG). The replication of HGG in the brain indicates that lentivectors causing tumors in the spinal cord are likely to do so in the brain as well. Successful implementation of this model requires precise stereotactic targeting to ensure a long trajectory that avoids ventricles (to prevent vector dilution) and sulci (to minimize inflammation). Avoiding vector reflux into the cranial subarachnoid space necessitates a trajectory longer than 1.5 cm and specific anti-reflux strategies, such as a stepped catheter design, post-infusion air bubble injection, pumped infusion rather than bolus injection, or creating a small cavity beyond the injection site by retracting the cannula to allow vector collection. Consequently, the critical elements of this model include precise targeting, effective anti-reflux strategies, the use of inbred animal strains, and the knockdown of specific tumor suppressors implicated in human tumors.

| Feature | Low-Grade Gliomas (LGG) | High-Grade Gliomas (HGG) |
|---|---|---|
| WHO Grades | Grade I (e.g., pilocytic | Grade III (e.g., anaplastic astrocytoma, |
| | astrocytoma), Grade II (e.g., | anaplastic oligodendroglioma), Grade |
| | diffuse astrocytoma, | IV (e.g., glioblastoma). |
| | oligodendroglioma). | |
| Growth Rate | Slow-growing. | Rapid-growing and highly aggressive. |
| Histological | Minimal atypia, low cellularity, no | High cellularity, nuclear atypia, mitotic |
| Features | necrosis or microvascular | activity, necrosis, and microvascular |
| | proliferation. | proliferation (especially in |
| | | glioblastoma). |
| Symptoms | May develop slowly; include | Develop quickly; include severe |
| | seizures, headaches, and focal | headaches, seizures, cognitive decline, |
| | neurological deficits. | and neurological deficits. |
| Prognosis | Better prognosis; survival can | Poor prognosis; survival is typically |
| | range from several years to | months to a few years, depending on |
| | decades with proper treatment. | grade and treatment. |
| Molecular | Frequently associated with IDH | IDH mutation may or may not be |
| Markers | mutations (IDH1/IDH2), ATRX | present. Glioblastomas often show |
| | mutations, and 1p/19q co- | EGFR amplification, PTEN mutations, or |
| | deletion. | TERT promoter mutations. |
| Imaging | Hypointense or isointense on T1- | Contrast enhancement on MRI |
| Features | weighted MRI, hyperintense on | (especially in glioblastomas, showing a |
| | T2/FLAIR without enhancement | ring-enhancing lesion with central |
| | (often). | necrosis). |
| Progression | LGG can transform into HGG over | HGGs are already aggressive and rarely |
| | time, a process called malignant | regress or downgrade. |
| | progression. | |

In some examples, disclosed herein is a large non-human mammal model of intracranial cancer. Large non-human mammal models include but are not limited to porcine (pig), ovine (sheep) or canine (dog) models. The large non-human mammal model of intracranial cancer comprising: an intracranial cancer engineered large non-human mammal with a combination of viral vectors encoding oncogenes or shRNA targeting tumor suppressor genes. Some exemplary viral vectors include but are not limited to recombinant adenoviral vectors, recombinant adeno-associated viral vectors, herpes simplex virus type 1 vectors, or lentiviral vectors. As used herein, some exemplary viral vectors encoding oncogenes include but are not limited to platelet-derived growth factor receptor alpha (PDGFRA), platelet-derived growth factor beta (PDGFRB), or mutated Harvey rat sarcoma viral oncogene (HRAS-G12V). As disclosed herein, in some examples, the PDGFRA, PDGFRB or HRAS-G12V is operably linked to an Ef1α promoter. As used herein, some exemplary viral vectors encoding shRNA include but are not limited to sh787 or sh944. Some exemplary viral vectors encoding shRNA targeting tumor suppressor genes include but are not limited to p53 (TP53), RB1 (retinoblastoma protein), PTEN, CDKN2A (p16), NF1 (neurofibromatosis type 1), or STK1. As disclosed herein, some exemplary viral vectors encoding shRNA targeting tumor suppressor genes include but are not limited to CDKN2A, PTEN or p53.

“Lentivirus” is a type of retrovirus that can integrate their genetic material into the host cell's DNA, allowing for sustained expression of the introduced genes. As used herein, engineering a lentivirus to carry oncogenes (genes that promote cancer growth), can induce tumor formation in the brain of an animal model when injected.

As used herein the following combinations of lentiviral vectors comprising oncogenes and shRNA targeting tumor suppressor genes, injected intraparenchymal in the brain of the non-human animal model to induce intracranial tumors: PDGFB+shP53, EGFR+shPTEN, PDGFB+H3K27M+shCDKN2A, and BRAF V600E+PDGFB+shP53.

| Vectors | Histopathological Findings |
|---|---|
| PDGFB + HRAS + shP53 | Glioblastoma |
| PDGFB + shP53 | Diffuse low-grade glioma |
| EGFR + shPTEN | Infiltrating glioma |
| EGFRVIII + shPTEN | Infiltrating glioma |
| PDGFB + H3K27M + shCDKN2A | Diffuse low-grade glioma |
| BRAF V600E + PDGFB + shP53 | Sarcoma |
| IDH1wt + PGFDB + shP53 | Potential glioblastoma |
| IDH1 R132H + PGFDB + shP53 | Potential low-grade glioma |

As used herein, the “BRAF V600E” mutation involves a substitution of valine (V) with glutamic acid (E) at position 600 in the BRAF gene. This alteration leads to continuous activation of the BRAF protein, resulting in uncontrolled cell proliferation and tumor development. This mutation is implicated in various cancers, including melanoma, colorectal cancer, non-small-cell lung cancer, and papillary thyroid carcinoma.

As used herein, the “H3K27M” mutation is a specific alteration in histone H3 proteins, where lysine (K) at position 27 is replaced by methionine (M). This mutation is predominantly associated with diffuse midline gliomas (DMGs), a category of aggressive brain tumors that primarily affect children and young adults.

As used herein, the “R132H” mutation is a specific mutation where the amino acid arginine (“R”) at position 132 in the IDH1 gene is replaced by histidine (“H”). This mutation leads to the accumulation of 2-hydroxyglutarate (2-HG), causing significant epigenetic and metabolic alterations and is a significant factor in determining prognosis in patients with gliomas.

As disclosed herein, the large non-human mammal model of intracranial cancer comprising: an intracranial cancer engineered large non-human mammal with a combination of viral vectors encoding oncogenes or shRNA targeting tumor suppressor genes produces the intracranial tumor, wherein the intracranial tumor biopsy comprises a population of intracranial cancer cells from the large non-human mammal model. In some examples, the large non-human mammal is a Gottingen minipig.

In some examples, each viral vector in the combination is encapsulated in a nanoparticle, a polymer, or a liposome.

In some examples, disclosed herein is a method of growing an intracranial tumor in a large non-human mammal model (Gottingen minipig), comprising: locating an intracranial delivery location in the large non-human mammal model using stereotactic neuronavigation; administering a combination of viral vectors encoding oncogenes and/or shRNA targeting tumor suppressor genes at the intracranial delivery location; avoiding vector reflux to sulci and the ventricular system, thereby inducing intracranial tumor. The method further comprises: obtaining a biopsy sample from the intracranial tumor; culturing cells from the biopsy sample; and evaluating intracranial tumor markers.

In some examples, the biopsy sample is obtained after 4 weeks of administering a combination of viral vectors in the large non-human mammal model.

In some examples, the combination of viral vectors comprises each viral vector titered at about >10², 10³, 10⁴, 10⁵, 10⁶, 10⁷, 10⁸, 10⁹, 10¹⁰, or 10¹¹ infectious units (IU)/ml. In some examples, the combination of viral vectors comprises each viral vector titered at about 10⁸ to about 10⁹ infectious units (IU)/ml. In some examples, the combination of viral vectors encoding oncogenes or shRNA targeting tumor suppressor genes is delivered concurrently or sequentially.

In some examples, the combination of viral vectors is administered by an intraparenchymal injection route.

In some examples, the volume of the combination of viral vectors administered in an injection range from about 10 μL, 15 μL, 20 L, 25 μL, 30 μL, 35 μL, 40 μL, 45 μL, 50 μL, 55 μL, 60 μL, or 65 μL. In some examples, the combination of viral vectors is administered in an injection volume ranging from about 10 μL to about 50 μL.

In some examples, the intracranial delivery location is supratentorial, wherein the supratentorial location comprises subcortical white matter.

In some examples, the neuronavigation comprises stereotactic targeting of the intracranial delivery location avoiding sulci and the ventricular system to minimize leaching of vector injection and maintain precision of targeting. “Stereotactic targeting” refers to a technique that uses imaging and special equipment to locate and treat abnormal areas in the body. Stereotactic surgery is a minimally invasive procedure that uses a 3D coordinate system to perform actions like biopsies, injections, and radiosurgery.

In some examples, the intracranial tumor markers comprise platelet-derived growth factor receptor alpha (PDGFRA), platelet-derived growth factor beta (PDGFRB), harvey rat sarcoma viral oncogene (HRAS), glial fibrillary acidic protein (GFAP), oligodendrocyte lineage transcription factor 2 (Olig2), neuron-glial antigen 2 (NG2), SRY-box transcription factor 2 (SOX2), NAD (P) H quinone dehydrogenase 1 (NQO1), or glutathione s-transferase pi 1 (GSTP1).

In some examples, the tumor shows histopathological features characteristic of high-grade glioma, including but not limited to parenchymal exophytic components, hypercellularity, atypical nuclei, mitotic indices, necrosis, hemorrhage, increased vascularity, or astrocytic morphology, elevated oxidative stress markers, increased cell proliferation, and increased inflammatory infiltrates.

In some examples, the tumor expresses markers associated with glioma, including GFAP, OLIG2, SOX2, PDGFRA, and CD68. Also disclosed herein, some tumor cells cultured from the animal cancer model exhibit increased proliferation compared to non-tumorigenic control cells and show immunopositivity for oncogenic markers.

In some examples, the system for creating and characterizing intracranial tumors in large non-human mammal models, comprises a lentiviral vector production system configured to produce replication-deficient vectors containing oncogenic or tumor-suppressor-targeting sequences. In some examples, the vectors are pseudotyped for targeted gene expression in the large non-human mammal brain. Disclosed herein the system for creating and characterizing intracranial tumors in large non-human mammal models also contains a stereotactic injection apparatus for administering the vector combination to a precise intracranial site in the animal and an imaging device configured to perform MRI scans for evaluating radiological characteristics of tumor formation. Further, the system comprises a histopathology and cell culture platform for analyzing biopsy samples obtained from the animal. In some examples, the system further comprises a cell culture system configured to support primary cell cultures derived from tumor biopsies and perform cell proliferation assays.

In some examples, the lentiviral vector production system is configured to produce vectors expressing at least one of PDGF-B, HRAS, or shRNA targeting TP53.

In some examples, the imaging device includes MRI equipment capable of conducting T1, T2, T2-FLAIR, and gradient echo sequences to visualize tumor contrast enhancement, peritumoral edema, necrosis, and mass effect.

In some examples, the immunopositive glioma markers include but are not limited to Platelet-Derived Growth Factor Receptor Alpha (PDGFRA), Platelet-Derived Growth Factor Beta (PDGFRB), Harvey Rat Sarcoma Viral Oncogene (HRAS), Glial Fibrillary Acidic Protein (GFAP), Oligodendrocyte Lineage Transcription Factor 2 (Olig2), Neuron-Glial Antigen 2 (NG2), SRY-Box Transcription Factor 2 (SOX2), and Platelet-Derived Growth Factor Receptor Alpha (PDGFRA), NAD (P) H Quinone Dehydrogenase 1 (NQO1), Glutathione S-Transferase Pi 1 (GSTP1), or CCAAT/Enhancer Binding Protein Beta (C/EBPβ).

Reference will now be made in detail to the examples of the invention, examples of which are illustrated in the drawings and the examples. This invention may, however, be embodied in many different forms and should not be construed as limited to the examples set forth herein.

Unless defined otherwise, all technical and scientific terms used herein have the same meaning as commonly understood to one of ordinary skill in the art to which this disclosure belongs.

### Terminology

Terms used throughout this application are to be construed with ordinary and typical meaning to those of ordinary skill in the art. However, Applicant desires that the following terms be given the particular definition as defined below.

As used herein, the article “a,” “an,” and “the” means “at least one,” unless the context in which the article is used clearly indicates otherwise.

“Administration” to a subject or “administering” includes any route of introducing or delivering to a subject an agent. Administration can be carried out by any suitable route, including oral, intravenous, intraperitoneal, intranasal, inhalation and the like. Administration includes self-administration and the administration by another.

The terms “about” and “approximately” are defined as being “close to” as understood by one of ordinary skill in the art. In one non-limiting embodiment, the terms are defined to be within 10%. In another non-limiting embodiment, the terms are defined to be within 5%. In still another non-limiting embodiment, the terms are defined to be within 1%.

The term “cancer” or “neoplasms” used herein meant to include all types of cancerous growths or oncogenic processes, metastatic tissues or malignantly transformed cells, tissues, or organs, irrespective of histopathologic type or stage of invasiveness. The terms “cancer” or “neoplasms” include malignancies of the various organ systems, such as malignancies affecting skin, brain, spinal cord, cervix, bladder, lung, breast, thyroid, lymphoid tissues, connecting tissues, gastrointestinal, and genito-urinary tracts, that include, but are not limited to, glioma, melanoma, lung cancer, breast cancer, cervical squamous cell carcinoma, bladder cancer, and soft tissue sarcoma. The term “cancer metastasis” has its general meaning in the art and refers to the spread of a tumor from one organ or part to another non-adjacent organ or part.

The term “comprising” and variations thereof as used herein is used synonymously with the term “including” and variations thereof and are open, non-limiting terms. Although the terms “comprising” and “including” have been used herein to describe various examples, the terms “consisting essentially of” and “consisting of” can be used in place of “comprising” and “including” to provide for more specific examples and are also disclosed.

A “composition” is intended to include a combination of active agent and another compound or composition, inert (for example, a detectable agent or label) or active, such as an adjuvant.

As used herein, the terms “determining,” “measuring,” and “assessing,” and “assaying” are used interchangeably and include both quantitative and qualitative determinations.

By the term “effective amount” of a therapeutic agent is meant a nontoxic but sufficient amount of a beneficial agent to provide the desired effect. The amount of beneficial agent that is “effective” will vary from subject to subject, depending on the age and general condition of the subject, the particular beneficial agent or agents, and the like. Thus, it is not always possible to specify an exact “effective amount.” However, an appropriate “effective” amount in any subject case may be determined by one of ordinary skill in the art using routine experimentation. Also, as used herein, and unless specifically stated otherwise, an “effective amount” of a beneficial can also refer to an amount covering both therapeutically effective amounts and prophylactically effective amounts.

An “effective amount” of a drug necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

As used herein the term “encoding” refers to the inherent property of specific sequences of nucleotides in a nucleic acid, to serve as templates for synthesis of other molecules having a defined sequence of nucleotides (i.e. rRNA, tRNA, other RNA molecules) or amino acids and the biological properties resulting therefrom.

The “fragments” or “functional fragments,” whether attached to other sequences or not, can include insertions, deletions, substitutions, or other selected modifications of particular regions or specific amino acids residues, provided the activity of the fragment is not significantly altered or impaired compared to the nonmodified peptide or protein. These modifications can provide for some additional property, such as to remove or add amino acids capable of disulfide bonding, to increase its bio-longevity, to alter its secretory characteristics, etc. In any case, the functional fragment must possess a bioactive property, such as antigen binding and antigen recognition.

The term “gene” or “gene sequence” refers to the coding sequence or control sequence, or fragments thereof. A gene may include any combination of coding sequence and control sequence, or fragments thereof. Thus, a “gene” as referred to herein may be all or part of a native gene. A polynucleotide sequence as referred to herein may be used interchangeably with the term “gene”, or may include any coding sequence, non-coding sequence or control sequence, fragments thereof, and combinations thereof. The term “gene” or “gene sequence” includes, for example, control sequences upstream of the coding sequence (for example, the ribosome binding site).

The term “isolating” as used herein refers to isolation from a biological sample, i.e., blood, plasma, tissues, exosomes, or cells. As used herein the term “isolated,” when used in the context of, e.g., a nucleic acid, refers to a nucleic acid of interest that is at least 60% free, at least 75% free, at least 90% free, at least 95% free, at least 98% free, and even at least 99% free from other components with which the nucleic acid is associated with prior to purification.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

The term “nucleic acid” refers to a natural or synthetic molecule comprising a single nucleotide or two or more nucleotides linked by a phosphate group at the 3′ position of one nucleotide to the 5′ end of another nucleotide. The nucleic acid is not limited by length, and thus the nucleic acid can include deoxyribonucleic acid (DNA) or ribonucleic acid (RNA). The term “oligonucleotide” denotes single- or double-stranded nucleotide multimers of from about 2 to up to about 100 nucleotides in length. Suitable oligonucleotides may be prepared by the phosphoramidite method described by Beaucage and Carruthers, Tetrahedron Lett., 22:1859-1862 (1981), or by the triester method according to Matteucci, et al., J. Am. Chem. Soc., 103:3185 (1981), both incorporated herein by reference, or by other chemical methods using either a commercial automated oligonucleotide synthesizer or VLSIPS™ technology. When oligonucleotides are referred to as “double-stranded,” it is understood by those of skill in the art that a pair of oligonucleotides exist in a hydrogen-bonded, helical array typically associated with, for example, DNA. In addition to the 100% complementary form of double-stranded oligonucleotides, the term “double-stranded,” as used herein is also meant to refer to those forms which include such structural features as bulges and loops, described more fully in such biochemistry texts as Stryer, Biochemistry, Third Ed., (1988), incorporated herein by reference for all purposes.

The term “polynucleotide” refers to a single or double stranded polymer composed of nucleotide monomers.

The term “polypeptide” refers to a compound made up of a single chain of D- or L-amino acids or a mixture of D- and L-amino acids joined by peptide bonds.

The terms “peptide,” “protein,” and “polypeptide” are used interchangeably to refer to a natural or synthetic molecule comprising two or more amino acids linked by the carboxyl group of one amino acid to the alpha amino group of another.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) nucleotide sequence identity is defined as the percentage of amino acids in a candidate sequence that are identical to the nucleotides in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

One example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990) J. Mol. Biol. 215:403-410). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P (N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01.

As used herein, the term “pharmaceutically acceptable” component can refer to a component that is not biologically or otherwise undesirable, i.e., the component may be incorporated into a pharmaceutical formulation of the invention and administered to a subject as described herein without causing any significant undesirable biological effects or interacting in a deleterious manner with any of the other components of the formulation in which it is contained. When the term “pharmaceutically acceptable” is used to refer to an excipient, it is generally implied that the component has met the required standards of toxicological and manufacturing testing or that it is included on the Inactive Ingredient Guide prepared by the U.S. Food and Drug Administration.

The term “subject” or “host” refers to any individual who is the target of administration or treatment. The subject can be a vertebrate, for example, a mammal. Thus, the subject can be a human or veterinary patient. The term “patient” refers to a subject under the treatment of a clinician, e.g., physician. The subject can be either male or female.

A control sample or a reference sample as described herein can be a sample from a healthy subject or sample, a wild-type subject or sample, or from populations thereof. A reference value can be used in place of a control or reference sample, which was previously obtained from a healthy subject or a group of healthy subjects or a wild-type subject or sample. A control sample or a reference sample can also be a sample with a known amount of a detectable compound or a spiked sample.

The term “tissue” refers to a group or layer of similarly specialized cells which together perform certain special functions. The term “tissue” is intended to include, blood, blood preparations such as plasma and serum, bones, joints, muscles, smooth muscles, lung tissues, and organs.

As used herein, the terms “treating” or “treatment” of a subject includes the administration of a drug to a subject with the purpose of preventing, curing, healing, alleviating, relieving, altering, remedying, ameliorating, improving, stabilizing or affecting a disease or disorder (e.g., a cancer), or a symptom of a disease or disorder. The terms “treating” and “treatment” can also refer to reduction in severity and/or frequency of symptoms, elimination of symptoms and/or underlying cause, prevention of the occurrence of symptoms and/or their underlying cause, and improvement or remediation of damage.

As used herein, a “therapeutically effective amount” of a therapeutic agent refers to an amount that is effective to achieve a desired therapeutic result, and a “prophylactically effective amount” of a therapeutic agent refers to an amount that is effective to prevent an unwanted physiological condition (e.g. cancer). Therapeutically effective and prophylactically effective amounts of a given therapeutic agent will typically vary with respect to factors such as the type and severity of the disorder or disease being treated and the age, gender, and weight of the subject.

The term “therapeutically effective amount” can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the drug and/or drug formulation to be administered (e.g., the potency of the therapeutic agent (drug), the concentration of drug in the formulation, and the like), and a variety of other factors that are appreciated by those of ordinary skill in the art.

In some examples, numbers expressing quantities of ingredients, properties such as molecular weight, reaction conditions, and so forth, used to describe and claim certain examples of the present disclosure are to be understood as being modified in some instances by the term “about.” In some examples, the term “about” is used to indicate that a value includes the standard deviation of the mean for the device or method being employed to determine the value. In some examples, the numerical parameters set forth in the written description and attached claims are approximations that can vary depending upon the desired properties sought to be obtained by a particular embodiment. In some examples, the numerical parameters should be construed in light of the number of reported significant digits and by applying ordinary rounding techniques. Notwithstanding that the numerical ranges and parameters setting forth the broad scope of some examples of the present disclosure are approximations, the numerical values set forth in the specific examples are reported as precisely as practicable. The numerical values presented in some examples of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

Throughout this application, various publications are referenced. The disclosures of these publications in their entirety are hereby incorporated by reference into this application in order to more fully describe the state of the art to which this pertains. The references disclosed are also individually and specifically incorporated by reference herein for the material contained in them that is discussed in the sentence in which the reference is relied upon.

### Molecular Engineering

The term “transfection,” as used herein, refers to the process of introducing nucleic acids into cells by non-viral methods. The term “transduction,” as used herein, refers to the process whereby foreign DNA is introduced into another cell via a viral vector.

The terms “heterologous DNA sequence”, “exogenous DNA segment”, or “heterologous nucleic acid,” as used herein, each refers to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling or cloning. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

An “expression vector”, otherwise known as an “expression construct”, is generally a plasmid or virus designed for gene expression in cells. The vector is used to introduce a specific gene into a target cell, and can commandeer the cell's mechanism for protein synthesis to produce the protein encoded by the gene. Expression vectors are the basic tools in biotechnology for the production of proteins. The vector is engineered to contain regulatory sequences that act as enhancer and/or promoter regions and lead to efficient transcription of the gene carried on the expression vector. The goal of a well-designed expression vector is the efficient production of protein, and this may be achieved by the production of significant amount of stable messenger RNA, which can then be translated into protein. The expression of a protein may be tightly controlled, and the protein is only produced in significant quantity, when necessary, through the use of an inducer, in some systems however the protein may be expressed constitutively. As described herein, Escherichia coli is used as the host for protein production, but other cell types may also be used.

In molecular biology, an “inducer” is a molecule that regulates gene expression. An inducer can function in two ways, such as:
(i) By disabling repressors. The gene is expressed because an inducer binds to the repressor. The binding of the inducer to the repressor prevents the repressor from binding to the operator. RNA polymerase can then begin to transcribe operon genes.
(ii) By binding to activators. Activators generally bind poorly to activator DNA sequences unless an inducer is present. An activator binds to an inducer and the complex binds to the activation sequence and activates target gene. Removing the inducer stops transcription. Because a small inducer molecule is required, the increased expression of the target gene is called induction.

Repressor proteins bind to the DNA strand and prevent RNA polymerase from being able to attach to the DNA and synthesize mRNA. Inducers bind to repressors, causing them to change shape and preventing them from binding to DNA. Therefore, they allow transcription, and thus gene expression, to take place.

For a gene to be expressed, its DNA sequence must be copied (in a process known as transcription) to make a smaller, mobile molecule called messenger RNA (mRNA), which carries the instructions for making a protein to the site where the protein is manufactured (in a process known as translation). Many different types of proteins can affect the level of gene expression by promoting or preventing transcription. In prokaryotes (such as bacteria), these proteins often act on a portion of DNA known as the operator at the beginning of the gene. The promoter is where RNA polymerase, the enzyme that copies the genetic sequence and synthesizes the mRNA, attaches to the DNA strand.

Some genes are modulated by activators, which have the opposite effect on gene expression as repressors. Inducers can also bind to activator proteins, allowing them to bind to the operator DNA where they promote RNA transcription. Ligands that bind to deactivate activator proteins are not, in the technical sense, classified as inducers, since they have the effect of preventing transcription.

A “promoter” is generally understood as a nucleic acid control sequence that directs transcription of a nucleic acid. An inducible promoter is generally understood as a promoter that mediates transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “ribosome binding site”, or “ribosomal binding site (RBS)”, refers to a sequence of nucleotides upstream of the start codon of an mRNA transcript that is responsible for the recruitment of a ribosome during the initiation of translation. Generally, RBS refers to bacterial sequences, although internal ribosome entry sites (IRES) have been described in mRNAs of eukaryotic cells or viruses that infect eukaryotes. Ribosome recruitment in eukaryotes is generally mediated by the 5′ cap present on eukaryotic mRNAs.

A “transcribable nucleic acid molecule” as used herein refers to any nucleic acid molecule capable of being transcribed into an RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10:0879697717; Ausubel et al. (2002) Short Protocols in Molecular Biology, 5th ed., Current Protocols, ISBN-10:0471250929; Sambrook and Russel (2001) Molecular Cloning: A Laboratory Manual, 3d ed., Cold Spring Harbor Laboratory Press, ISBN-10:0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position +1. With respect to this site all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A construct of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal, or a plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation. Design, generation, and testing of the variant nucleotides, and their encoded polypeptides, having the above-required percent identities and retaining a required activity of the expressed protein is within the skill of the art. For example, directed evolution and rapid isolation of mutants can be according to methods described in references including, but not limited to, Link et al. (2007) Nature Reviews 5 (9), 680-688; Sanger et al. (1991) Gene 97 (1), 119-123; Ghadessy et al. (2001) Proc Natl Acad Sci USA 98 (8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2, or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=XN100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A. For example, the percent identity can be at least 80% or about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

Substitution refers to the replacement of one amino acid with another amino acid in a protein or the replacement of one nucleotide with another in DNA or RNA. Insertion refers to the insertion of one or more amino acids in a protein or the insertion of one or more nucleotides with another in DNA or RNA. Deletion refers to the deletion of one or more amino acids in a protein or the deletion of one or more nucleotides with another in DNA or RNA. Generally, substitutions, insertions, or deletions can be made at any position so long as the required activity is retained. So-called conservative exchanges can be carried out in which the amino acid which is replaced has a similar property as the original amino acid, for example, the exchange of Glu by Asp, Gln by Asn, Val by Ile, Leu by Ile, and Ser by Thr. For example, amino acids with similar properties can be Aliphatic amino acids (e.g., Glycine, Alanine, Valine, Leucine, Isoleucine); hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. An amino acid sequence can be modulated with the help of art-known computer simulation programs that can produce a polypeptide with, for example, improved activity or altered regulation. On the basis of these artificially generated polypeptide sequences, a corresponding nucleic acid molecule coding for such a modulated polypeptide can be synthesized in-vitro using the specific codon-usage of the desired host cell.

Host cells can be transformed using a variety of standard techniques known to the art (see e.g., Sambrook and Russel (2006) Condensed Protocols from Molecular Cloning: A Laboratory Manual, Cold Spring Harbor Laboratory Press, ISBN-10:0879697717; Ausubel et al. (2002) Short Protocols in Molecular Biology, 5th ed., Current Protocols, ISBN-10:0471250929; Sambrook and Russel (2001) Molecular Cloning: A Laboratory Manual, 3d ed., Cold Spring Harbor Laboratory Press, ISBN-10:0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transformed cells can be selected and propagated to provide recombinant host cells that comprise the expression vector stably integrated in the host cell genome.

Exemplary nucleic acids that may be introduced to a host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA that is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Methods of down-regulation or silence genes are known in art. For example, expressed protein activity can be downregulated or eliminated using antisense oligonucleotides (ASOs), protein aptamers, nucleotide aptamers, and RNA interference (RNAi) (e.g., small interfering RNAs (siRNA), short hairpin RNA (shRNA), and micro RNAs (miRNA) (see e.g., Rinaldi and Wood (2017) Nature Reviews Neurology 14, describing ASO therapies; Fanning and Symonds (2006) Handb Exp Pharmacol. 173, 289-303G, describing hammerhead ribozymes and small hairpin RNA; Helene, et al. (1992) Ann. N.Y. Acad. Sci. 660, 27-36; Maher (1992) Bioassays 14 (12): 807-15, describing targeting deoxyribonucleotide sequences; Lee et al. (2006) Curr Opin Chem Biol. 10, 1-8, describing aptamers; Reynolds et al. (2004) Nature Biotechnology 22 (3), 326-330, describing RNAi; Pushparaj and Melendez (2006) Clinical and Experimental Pharmacology and Physiology 33 (5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinformatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

As would be apparent, the sequencing may be done using a next generation sequencing platform, e.g., Illumina's reversible terminator method, Roche's pyrosequencing method, Life Technologies' sequencing by ligation (the SOLID platform) or Life Technologies' Ion Torrent platform, etc. Examples of such methods are described in the following references: Margulies et al (Nature 2005 437:376-80); Ronaghi et al (Analytical Biochemistry 1996 242:84-9); Shendure (Science 2005 309:1728); Imelfort et al (Brief Bioinform. 2009 10:609-18); Fox et al (Methods Mol Biol. 2009; 553:79-108); Appleby et al (Methods Mol Biol. 2009; 513:19-39) and Morozova (Genomics. 2008 92:255-64), which are incorporated by reference for the general descriptions of the methods and the particular steps of the methods, including all starting products, reagents, and final products for each of the steps. In other examples, the sequencing may be done using nanopore sequencing (e.g. as described in Soni et al Clin Chem 53:1996-2001 2007, or as described by Oxford Nanopore Technologies).

### Screening

Also provided are screening methods using the models and cells as described herein. The subject methods find use in the screening of a variety of different candidate molecules (e.g., potentially therapeutic candidate molecules). Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 MW, or less than about 1000 MW, or less than about 800 MW) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl, or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules; and electronic libraries of commercial compounds provided by vendors, for example, ChemBridge, Princeton BioMolecular, Ambinter SARL, Enamine, ASDI, Life Chemicals, etc.).

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character xlogP of about −2 to about 4) (see e.g., Angewante (1999) Chemie Int. ed. Engl. 24, 3943-3948). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character xlogP of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopoeia. While it is not required for drugs to meet all, or even any, of these characterizations, it is far more likely for a drug candidate to meet with clinical success if it is drug-like.

Several of these “drug-like” characteristics have been summarized into the four rules of Lipinski (generally known as the “rules of fives” because of the prevalence of the number among them). While these rules generally relate to oral absorption and are used to predict the bioavailability of a compound during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) a weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8A to about 15A.

In some examples, the anticancer agent is selected from the group consisting of cordycepin, fenretinide, Zyclara, vemurafenib (Zelboraf®), dabrafenib (Tafinlar™), encorafenib (Braftovi™), pembrolizumab (Keytruda), nivolumab (Opdivo), Anthracyclines, Taxanes, 5-fluorouracil (5-FU), Cyclophosphamide (Cytoxan), Carboplatin (Paraplatin), cisplatin, carboplatin, Vinorelbine (Navelbine), Capecitabine (Xeloda), Gemcitabine (Gemzar), Ixabepilone (Ixempra), Eribulin (Halaven), Fulvestrant (Faslodex), Letrozole (Femara), Anastrozole (Arimidex), exemestane (Aromasin), Trastuzumab (Herceptin), Pertuzumab (Perjeta), Ado-trastuzumab emtansine, Lapatinib (Tykerb), Neratinib (Nerlynx), Everolimus (Afinitor), Olaparib (Lynparza), talazoparib (Talzenna), Alpelisib (Piqray), Atezolizumab (Tecentriq), Paclitaxel (Taxol), Albumin-bound paclitaxel (nab-paclitaxel, Abraxane), Docetaxel (Taxotere), Etoposide (VP-16), Pemetrexed (Alimta), Bevacizumab (Avastin), Ramucirumab (Cyramza), ifosfamide (Ifex™), irinotecan (Camptosar™), mitomycin, doxorubicin (Adriamycin), methotrexate, vinblastine (CMV), durvalumab (Imfinzi™), avelumab (Bavencio™), Erdafitinib (Balversa), dacarbazine (DTIC), epirubicin, temozolomide (Temodar™), gemcitabine (Gemzar™), trabectedin (Yondelis™), and Pazopanib (Votrient).

### Gene Therapy and Genome Editing

Gene therapies can include inserting a functional gene with a viral vector. Gene therapies for cancers are rapidly advancing. There has recently been an improved landscape for gene therapies. For example, in the first quarter of 2019, there were 372 ongoing gene therapy clinical trials (Alliance for Regenerative Medicine, May 9, 2019).

Any vector known in art can be used. For example, the vector can be a viral vector selected from retrovirus, lentivirus, herpes, adenovirus, adeno-associated virus (AAV), rabies, Ebola, lentivirus, or hybrids thereof.

Gene Therapy Strategies

| | |
|---|---|
| | Associated experimental |
| Strategy | models |

Viral Vectors

| | | |
|---|---|---|
| Retrovisuses | Retroviruses are RNA viruses | Murine model of MPS [symbol] |
| | transcribing their single-stranded | [symbol] model of MPS [symbol] |
| | genome into a double-stranded | |
| | DNA copy, which can integrate | |
| | into host chromosome | |
| Adenoviruses (Ad) | Ad can tranfect a variety of | Murine model of [symbol] Fabry, |
| | quiescent and proliferating | [symbol] diseases, |
| | cell types from various species | [symbol] |
| | and can mediate | and MPS [symbol] |
| Adeno-associated | Recombinant AAV vectors | Musing models of [symbol] , Fabry |
| Viruses (AAV) | contain no viral DNA and can | diseases, [symbol] |
| | carry ~4.7 kb of foreign | Krabbe disease Metachromatic |
| | transgenic material. They | [symbol] |
| | are replication defective and can | [symbol] |
| | replicate only while | MPSVI, MPS VB, CLN1, CLN2, |
| | coinfecting with a helper virus | CLN3, CLN5, CLN6 |

Non-viral vectors

| | | |
|---|---|---|
| plasmid DNA | pDNA has many desired | Mouse model of Fabry disease |
| (pDNA) | characteristics as a gene | |
| | therapy vector; there are no limits | |
| | on the size or genetic | |
| | constitution of DNA, it is relatively | |
| | inexpensive to supply, | |
| | and unline viruses, antibodies are | |
| | not generated against DNA in normal | |
| | individuals | |
| RNAi | RNAi is a powerful tool for gene | Transgenic mouse strain |
| | specific silencing that | Mouse models of acute liver |
| | could be useful as an enzyme | failure |
| | reduction therapy or | Mice with hepatitis B virus |
| | means to promote read-through | Fabry mouse |
| | of a premature stop codon | |

[symbol]

indicates data missing or illegible when filed

Gene therapy can allow for the constant delivery of the enzyme directly to target organs and eliminates the need for weekly infusions. Also, correction of a few cells could lead to the enzyme being secreted into the circulation and taken up by their neighboring cells (cross-correction), resulting in widespread correction of the biochemical defects. As such, the number of cells that must be modified with a gene transfer vector is relatively low.

Genetic modification can be performed either ex vivo or in vivo. The ex vivo strategy is based on the modification of cells in culture and transplantation of the modified cell into a patient. Cells that are most commonly considered therapeutic targets for monogenic diseases are stem cells. Advances in the collection and isolation of these cells from a variety of sources have promoted autologous gene therapy as a viable option.

The use of endonucleases for targeted genome editing can solve the limitations presented by the usual gene therapy protocols. These enzymes are custom molecular scissors, allowing cutting DNA into well-defined, perfectly specified pieces, in virtually all cell types. Moreover, they can be delivered to the cells by plasmids that transiently express the nucleases, or by transcribed RNA, avoiding the use of viruses.

### Administration

Agents and compositions described herein can be administered according to methods described herein in a variety of ways known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, intraparenchymal, epidural, intrathecal, ophthalmic, transdermal, buccal, and rectal.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 pm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10:0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency; improve taste of the product; or improve shelf life of the product.

### Therapeutic Methods

Also provided is a process of treating, preventing, or reversing cancer (e.g., glioblastoma) in a subject in need of administration of a therapeutically effective amount of an agent, so as to substantially inhibit cancer, slow the progress of cancer, or limit the development of cancer.

Methods described herein are generally performed on a subject in need thereof. A subject in need of the therapeutic methods described herein can be a subject having, diagnosed with, suspected of having, or at risk for developing cancer. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, shecp, pigs, mice, rats, monkeys, hamsters, guinea pigs, and humans or chickens. For example, the subject can be a human subject.

Generally, a safe and effective amount of an agent is, for example, an amount that would cause the desired therapeutic effect in a subject while minimizing undesired side effects. In various examples, an effective amount of an agent described herein can substantially inhibit cancer, slow the progress of cancer, or limit the development of cancer.

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration.

When used in the treatments described herein, a therapeutically effective amount of an agent can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to substantially inhibit cancer, slow the progress of cancer, or limit the development of cancer.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; the activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Shamel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing, reversing, or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or a physician.

Administration of an agent can occur as a single event or over a time course of treatment. For example, an agent can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to or before, concurrent with, or after conventional treatment modalities for cancer. An agent can be administered simultaneously or sequentially with another agent, such as an antibiotic, an anti-inflammatory, or another agent. For example, an agent can be administered simultaneously with another agent, such as an antibiotic or an anti-inflammatory. Simultaneous administration can occur through the administration of separate compositions, each containing one or more of agents, anti-cancer agent, antibiotic, an anti-inflammatory, or another agent. Simultaneous administration can occur through administration of one composition containing two or more agents, anti-cancer agent, antibiotic, anti-inflammatory, or another agent. An agent can be administered sequentially with an anti-cancer agent, an antibiotic, an anti-inflammatory, or another agent. For example, an agent can be administered before or after administration of an anti-cancer agent, an antibiotic, an anti-inflammatory, or another agent.

Active compounds are administered at a therapeutically effective dosage sufficient to treat a condition associated with a condition in a patient. For example, the efficacy of a compound can be evaluated in an animal model system that may be predictive of efficacy in treating the disease in a human or another animal, such as the model systems shown in the examples and drawings.

An effective dose range of a therapeutic can be extrapolated from effective doses determined in animal studies for a variety of different animals. In general, a human equivalent dose (HED) in mg/kg can be calculated in accordance with the following formula (see e.g., Reagan-Shaw et al., FASEB J., 22 (3): 659-661, 2008, which is incorporated herein by reference):

HED(mg/kg)=Animal dose(mg/kg)×(Animal Kₘ/Human Kₘ)

Use of the Kₘ factors in conversion results in more accurate HED values, which are based on body surface area (BSA) rather than only on body mass. Kₘ values for humans and various animals are well known. For example, the Kₘ for an average 60 kg human (with a BSA of 1.6 m²) is 37, whereas a 20 kg child (BSA 0.8 m²) would have a Kₘ of 25. Kₘ for some relevant animal models is also well known, including: mice Kₘ of 3 (given a weight of 0.02 kg and BSA of 0.007); hamster Kₘ of 5 (given a weight of 0.08 kg and BSA of 0.02); rat Kₘ of 6 (given a weight of 0.15 kg and BSA of 0.025) and monkey Kₘ of 12 (given a weight of 3 kg and BSA of 0.24).

Precise amounts of the therapeutic composition depend on the judgment of the practitioner and are peculiar to each individual. Nonetheless, a calculated HED dose provides a general guide. Other factors affecting the dose include the physical and clinical state of the patient, the route of administration, the intended goal of treatment, and the potency, stability, and toxicity of the particular therapeutic formulation.

The actual dosage amount of a compound of the present disclosure or composition comprising a compound of the present disclosure administered to a subject may be determined by physical and physiological factors such as type of animal treated, age, sex, body weight, severity of condition, the type of disease being treated, previous or concurrent therapeutic interventions, idiopathy of the subject and on the route of administration. These factors may be determined by a skilled artisan. The practitioner responsible for administration will typically determine the concentration of active ingredient(s) in a composition and appropriate dose(s) for the individual subject. The dosage may be adjusted by the individual physician in the event of any complication.

### Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Md., 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutically active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce dosage frequency. Controlled-release preparations can also be used to affect the time of onset of action or other characteristics, such as blood levels of the agent, and consequently, affect the occurrence of side effects. Controlled-release preparations may be designed to initially release an amount of an agent(s) that produces the desired therapeutic effect, and gradually and continually release other amounts of the agent to maintain the level of the therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide the subject other therapies known to be efficacious for treatment of the disease, disorder, or condition.

### EXAMPLES

The following examples are set forth below to illustrate the compounds, systems, methods, and results according to the disclosed subject matter. These examples are not intended to be inclusive of all examples of the subject matter disclosed herein, but rather to illustrate representative methods and results. These examples are not intended to exclude equivalents and variations of the present invention which are apparent to one skilled in the art.

### Example 1 Shows Animals are Asymptomatic with a Decreased Volume of Injection by a 4-Week Endpoint

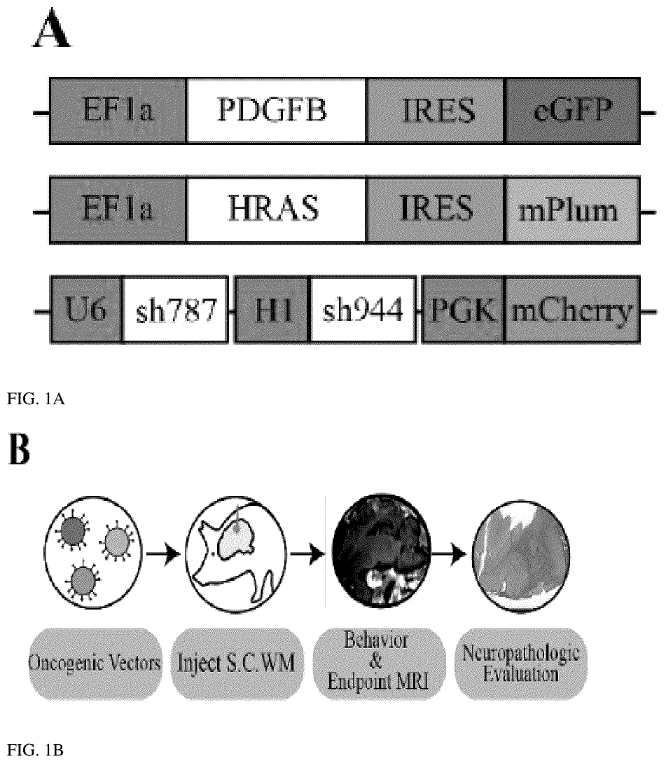

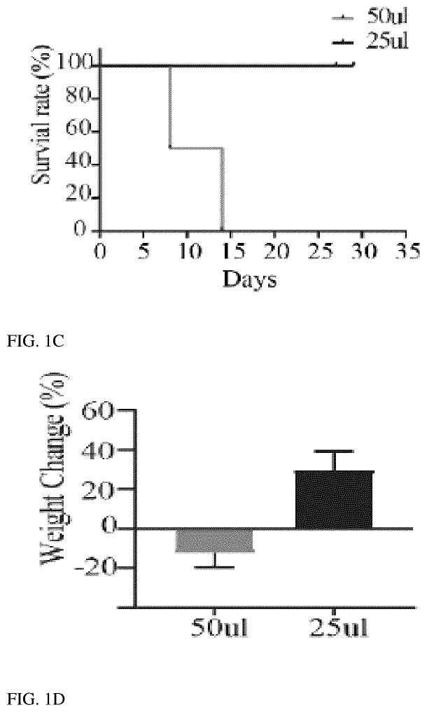

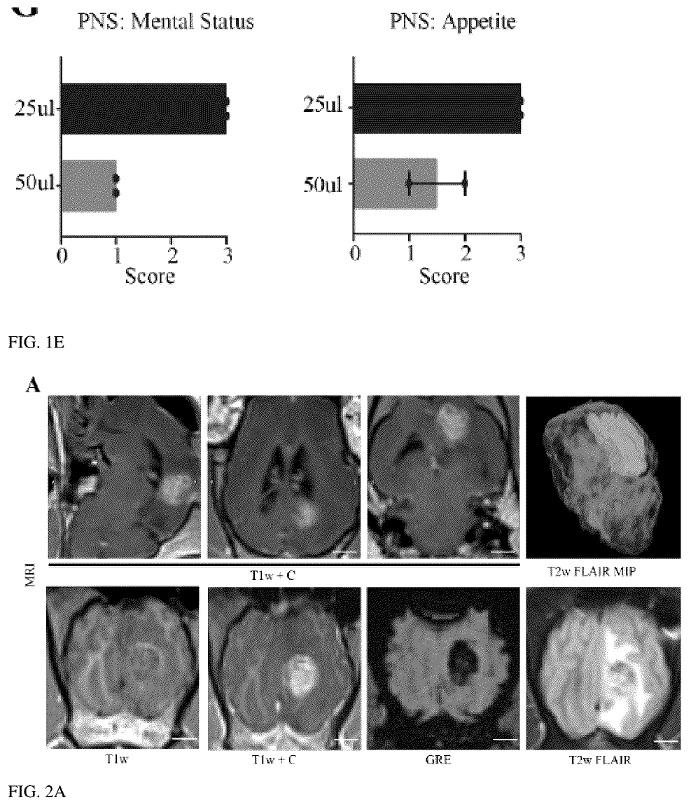

In order to elucidate a feasible volume of injection in our pilot study, we conducted two groups of animals at 50 μl and 25 μl injections. Two animals (N=2/2) that received 50 μl injections developed decreased appetite, neurologic decline, and weight loss corresponding to a decline in PNS score at endpoint. One animal reached endpoint by the end of post-operative week 1 and exhibited a generalized tonic-colonic seizure. The second animal reached endpoint by the end of post-operative week 2, presenting with localized seizures as facial automatisms, inappetence, emesis, and neurologic decline. Two animals at the 25 μl dose were conducted. These animals (N=2/2) were asymptomatic until the pre-determined 4-week endpoint, with no signs of weight loss, cachexia, or neurologic symptoms. Summary behavioral findings and survival are presented in FIGS. 1E, 1F and 1G.

### Example 2 Shows Injections to Subcortical Structures Produce Highly Penetrant Lesions

In order to maximize experimental yield, we elected to perform bilateral injections on different subcortical targets. Four animals underwent surgery as described, targeting the thalamus, subventricular zone (SVZ), or deep subcortical white matter (SWM), for a total of 8 injections. Over a period of up to 4 weeks, 4/4 animals reached humane endpoint due to depressed neurobehavioral score, inappetence, or seizures (mean time to endpoint 25 days±3.2). Another three animals underwent surgery targeting the deep nuclei, for a total of 3 injections. On gross pathology (FIG. 6) 8/8 injections resulted in invasive mass forming lesions at pre-planned targets. All lesions demonstrated punctate hemorrhage and gross hypervascularity. 6/8 lesions exhibited gross parenchymal hemorrhage with frank clot apparent on necropsy.

### Example 3 Demonstrates Histopathological and Immunohistochemical Characterization

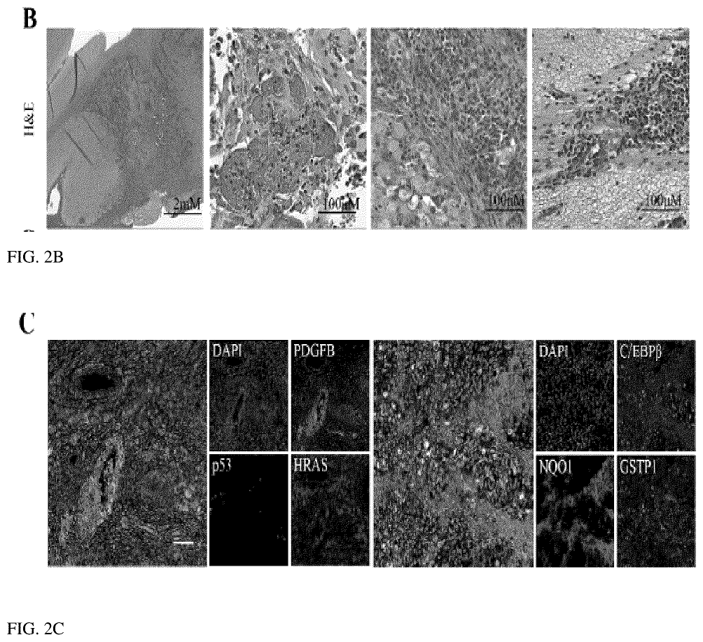

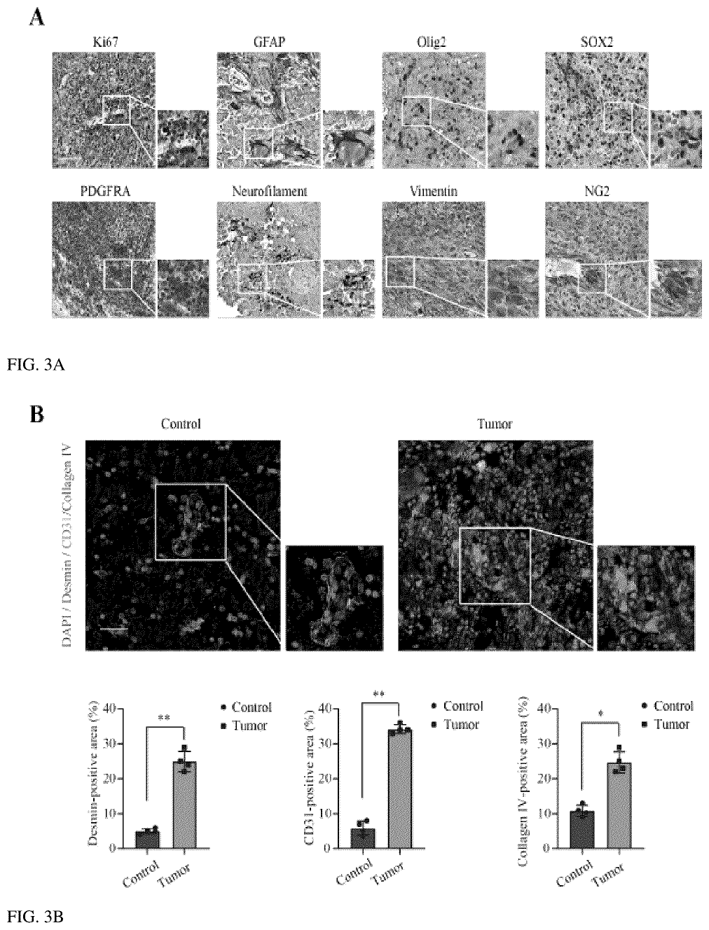

On H & E staining at low magnification, the tumors demonstrated cellular regions showing hyperchromasia with diffuse invasion of grey and white matter. Cortical spread of the lesion was observed confluent with the parenchymal lesion (FIG. 2B). At high magnification at sites distal to the injection, tumor invasion was observed along white matter tracts. Histopathologic features at high magnification included: high cellularity with epithelioid and fibrillary astrocytic morphology, microvascular proliferation, necrosis, thrombosed blood vessels, and invasion along the tumor border into surrounding normal parenchyma, consistent with a high-grade glioma. In addition, a significant degree of leptomeningeal inflammatory infiltrate was noted in the surrounding region (FIG. 2B). Immunofluorescent staining demonstrated sparse P53 and strong PDGFB and HRAS immunopositivity (FIG. 2C, left panel). In addition, our previously work demonstrated that C/EBPβ mediates NQO1 and GSTP1 anti-oxidative reductases expression in glioblastoma, promoting brain tumor proliferation. Thus, we stained with for C/EBPβ, NQO1, GSTP1, and we found that all of them were highly immunopositive in tumor tissue, which indicated that redox homeostasis in our model exists (FIG. 2C, right panel). Characterization of glioma markers in the tumor found GFAP, OLIG2, SOX2, NG2, and PDGFRA immunopositivity. Vimentin staining was grossly immunopositive, and neurofilament staining demonstrated presence of background parenchyma within the tumor bulk. (FIG. 3A). Ki-67 proliferative index was highly positive across animals, with a mean proliferative index of 44.1% (SD: ±13.6). Immunofluorescence staining was conducted to examine the tumor microenvironment further in comparison to control tissue, illustrating significantly increased Desmin, CD31, and Type IV collagen (FIG. 3B). The results indicated that tumor tissue showed an extensive and irregular endothelial cell/pericyte margin with an unbalanced basement membrane structure compared to the control tissue, which showed morphologic changes in the tumor microenvironment.

### Example 4 Demonstrates MRI at Endpoint Demonstrates Lesions with Radiologic Features Consistent with High-Grade Glioma

At endpoint, animals underwent MRI imaging prior to euthanasia and necropsy. In our experiments, three animals (N=¾) underwent MRI imaging due to the onset of seizure in the 50 μl group and need for euthanasia at veterinary direction. Endpoint scans demonstrated the presence of T1w contrast enhancing mass forming lesions with marked peri-lesional edema in both groups (FIG. 2A, T1w+Contrast, T2-FLAIR MIP). Tumor features across animals included T1w contrast enhancement (N=3/3), peri-tumoral edema on T2-FLAIR (N=3/3), and hemorrhage on GRE (N=⅔). Mass effect was noted in all animals (N=3/3) with compression of the posterior horn of the lateral ventricle at a mean 4.33 mm (±1.06) measured on coronal multiplanar reconstruction. Tumor dimensions included a mean maximal diameter of 11.7 mm (±2.93) and mean volume of 713.7 mm³ (±537.3).

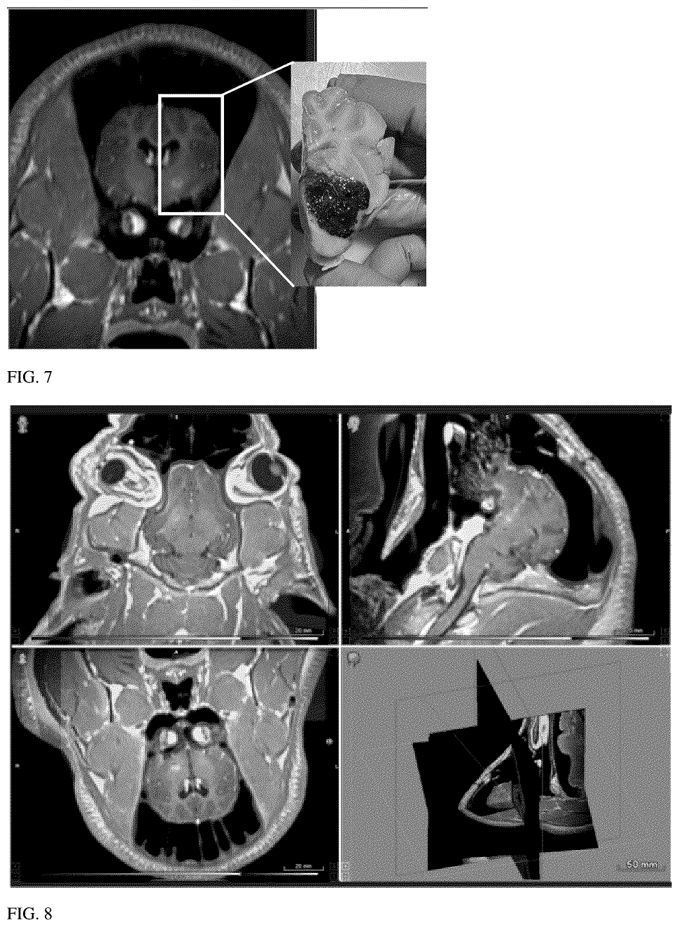

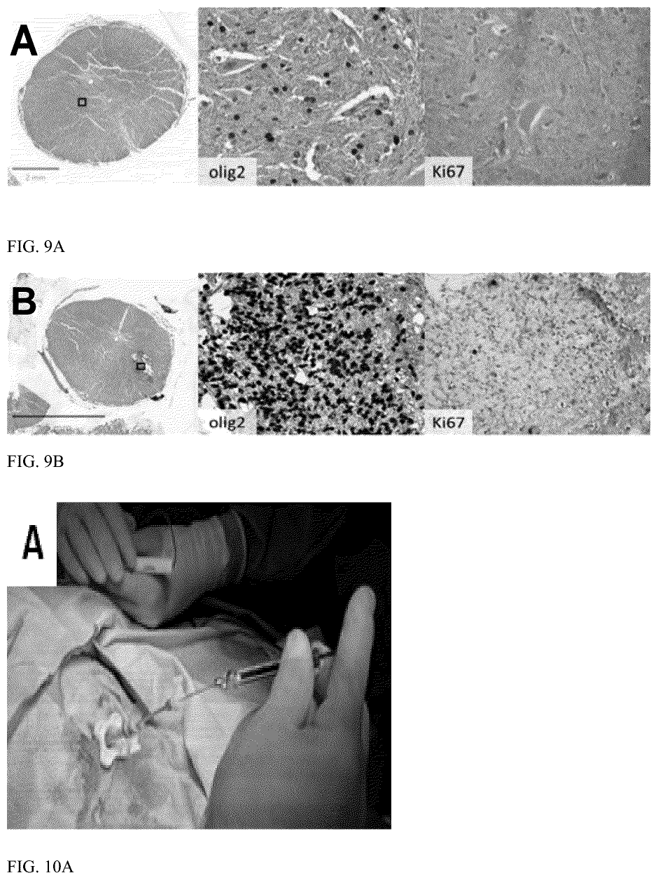

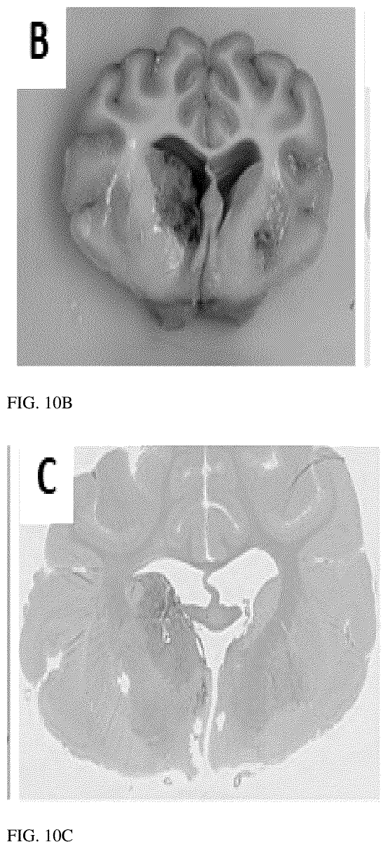

In another experiment only one animal underwent MRI scan at humane endpoint. On T1 weighted imaging, a hypointense region can be appreciated with mass effect causing effacement of the right lateral ventricle (FIG. 7). Heterogenous gadolinium enhancement (T1 Post) and significant punctate signal voids indicative of intratumoral hemorrhage (GRE) are seen at both injection sites. Gross peritumoral edema is present on T2 (T2 FLAIR). Overall, findings consistent with a high-grade primary neoplasm.

### Example 5 Shows the Establishment of Porcine Glioma Tissue Culture

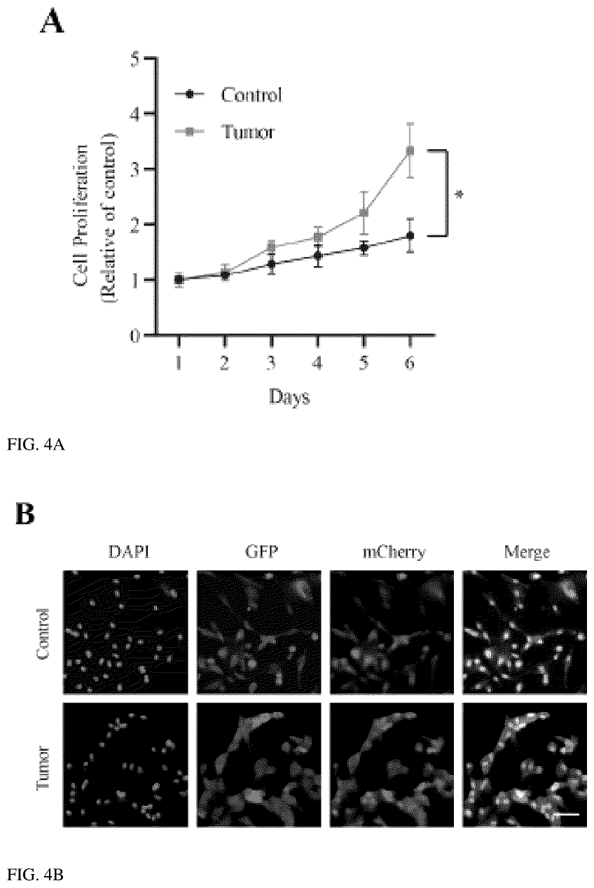

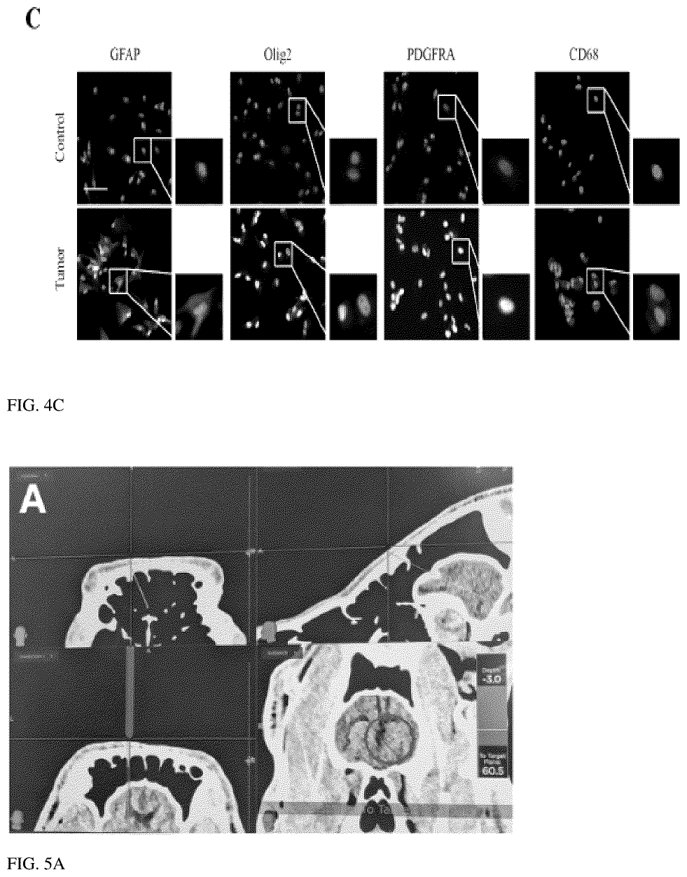

Tumor and control biopsies were cultured at the time of necropsy. Compared to control, the tumor cells demonstrated greater than 3 times the proliferative rate by culture day 6 (FIG. 4A). As expected, immunofluorescent staining for oncogenic and control vector reporters were found to be immunopositive in both control and tumor cells (FIG. 4B). Tumor cells were strongly immunopositive for GFAP, OLIG2, PDGFRA, and CD68 (FIG. 4C).

### Methods

### Lentiviral Vectors

Lentiviral vectors were designed to target the RTK/RAS/PI3K and TP53 pathways (Table 1 and Table 2). Vectors were designed separately to maintain transduction efficiency and in accordance with biosafety requirements as previously described in detail. Briefly, all viral vectors were VSV-G pseudotyped replication deficient lentiviral systems. PDGF-B-IRES-eGFP and HRASG12V-IRES-mPlum vectors were produced on a pCDH backbone with Ef1α promoter. shRNA-P53 vectors utilized a pLKO1 backbone and expressed shRNA targeting porcine p53 including sh787 and sh944 under H1 and U6 promoters. All vectors were thawed and combined into equal parts immediately prior to inoculation. Viral vectors were obtained from the Emory Viral Vector Core.

TABLE 1
VSV-G pseudotyped, third-generation, replication
deficient lentiviral vectors. Titers were at about 10⁸ to
about 10⁹ infectious units (IU)/ml.

| Vector | Backbone | Promoter(s) | Transgene(s) |
|---|---|---|---|
| 1 | pCDH | EF1α | PDGFB-IRES-eGFP |
| 2 | pCDH | EF1α | HRASG12V-IRES-mPlum |
| 3 | pLKO1 | U6, H1, PGK | shRNA-P53, mCherry |

TABLE 2
Combinations of viral vectors and histological findings

| Vectors | Histopathological Findings |
|---|---|
| PDGFB + HRAS + shP53 | Glioblastoma |
| PDGFB + shP53 | Diffuse low-grade glioma |
| EGFR + shPTEN | Infiltrating glioma |
| EGFRVIII + shPTEN | Infiltrating glioma |
| PDGFB + H3K27M + shCDKN2A | Diffuse low-grade glioma |
| BRAF V600E + PDGFB + shP53 | Sarcoma |
| IDH1WT + PGFDB + shP53 | Potential glioblastoma |
| IDH1 R132H + PGFDB + shP53 | Potential low-grade glioma |

### Animals, Physical Examination, and Behavior

Seven sexually matured female and male Gottingen minipigs, weighing approximately 20-30 Kgs, from Marshall BioResources, North Rose, NY, were utilized in this study. Routinely physical examinations were performed to monitor clinical general well-being and neurobehavioral deficits. All animals underwent modified Tarlov-motor scoring (mTS) and porcine neurobehavioral scoring (PNS). All experiments were conducted with adherence to approved protocols set forth by the Institutional Animal Care and Use Committee (IACUC), the Emory Division of Animal Resources (DAR) and T3 Labs (Translational Testing and Training Laboratories). Following lentiviral inoculation, animals were treated as ABSL-2 for 72 hours, per recommendation from both Institutional Biosafety Committees (IBC).

### Computed Tomography (CT)

Forty-eight hours prior to surgery, with the animal under general anesthesia, in prone position and with the head immobilized, a head CT scan was performed using a Siemens Biograph Vision 600 PET/CT scanner. The scan images were then reconstructed in coronal, axial, and sagittal planes using the ADMIRE 5 reconstruction methodology and uploaded to the Medtronic StealthStation™ S8 surgical navigation system (Medtronic, Inc., Minneapolis, MN, USA). While fiducial screws could have been placed to improve acquisition and reconstruction of the volumetric brain, the elongated and irregular swine skull provides adequate unique points to achieve registration with the StealthSystem EM Tracer Pointer.

### Peri- and Post-Operative Steroid and Anti-Seizure Regimens

Animals received peri- and post-operative anti-inflammatory (8-day course of intramuscular Dexamethasone, 0.2 mg/Kg) and seizure prophylaxis (7-day course of oral Levetiracetam, 10 mg/Kg, three times a day) treatments. These agents were administered to reduce the risk of intracerebral edema, inflammation, and seizures following craniotomy, as well as potential innate immune response against the lentiviral vectors.

### Registration and Trajectory Planning

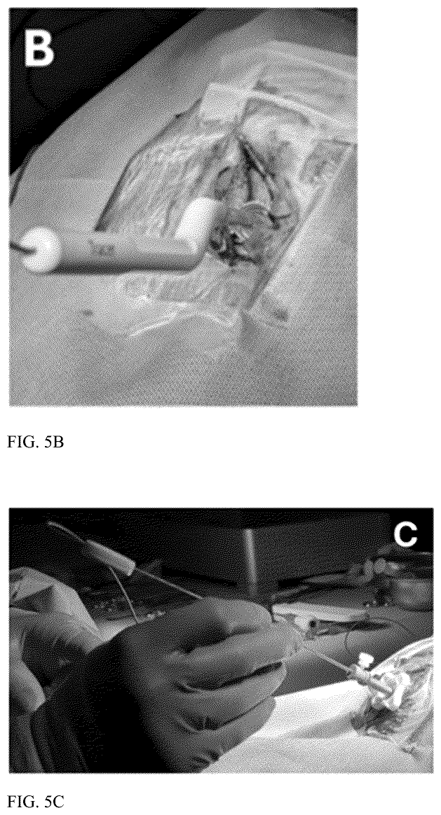

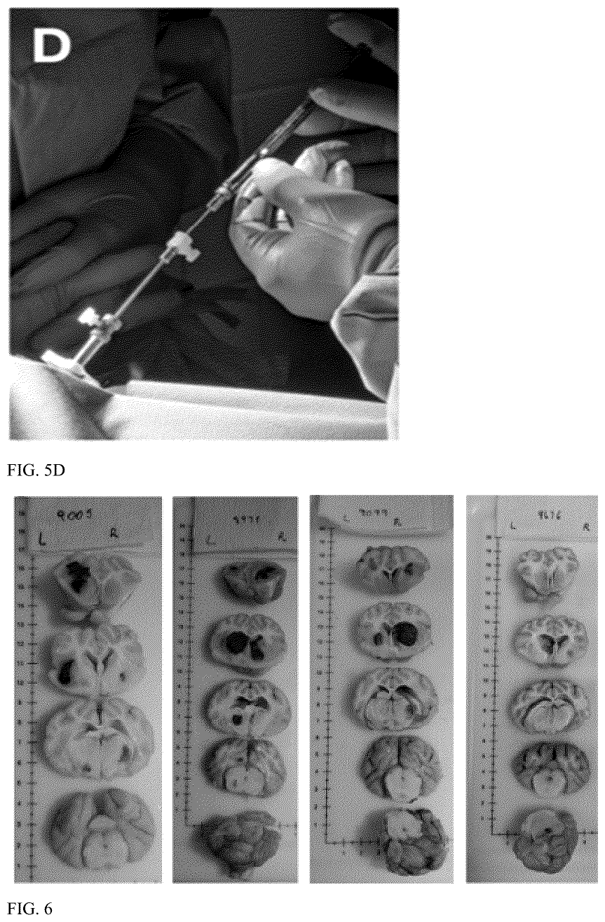

Under general anesthesia, with the animal in prone position and a StealthStation Flat Emitter placed beneath the patient's head, initial registration was obtained. Briefly, an EM (non-invasive) patient tracker was sutured on the proximal snout and a non-sterile handheld EM Tracer Pointer was used for surface registration. Planned trajectories targeting the thalamus, SVZ, or deep subcortical white matter for bilateral injections or thalamus alone for unilateral injections were then obtained (FIG. 5A). Care was taken to avoid sulci and the ventricular system to minimize leeching of vector injection and maintain precision of targeting.

### Surgical Technique

With the surgical field draped and prepared in a standard sterile manner, an S shaped ˜10 cm incision was made over the skull entry point predetermined during trajectory planning. Following careful dissection of the scalp layers, a ˜6 mm burr hole was made at the entry point in the outer table of the skull until the frontal sinus was entered and the base of a Stealth Navigus Frameless biopsy trajectory guide kit was screw mounted over the entry point burr hole. Next, using a sterile EM Tracer Pointer (FIG. 5B), a second burr hole was made in the inner table of the skull along the preselected trajectory until dura was visualized.

After completion of craniotomies, the EM Tracer Pointer was applied one more time to match the desired trajectory to within 1-2 mm of stereotactic planning. At this point, a bolus of methylprednisolone was administered to prevent edema prior to dural entry (125 mg, IV). Using a 14-gauge spinal needle as a guide aid placed into the biopsy trajectory guide tube, a durotomy was performed, and the EM stylet passed down the biopsy trajectory guide tube at the pre-determined depth (FIG. 5C). The EM stylet was removed and replaced with a custom introducer cannula (OD 1.2 mm) and stylet (OD 0.7 mm). A C-clamp was placed around the cannula to ensure accurate depth for needle introduction. A 22-gauge 8 in. long blunt needle connected to a gastight Hamilton syringe (Hamilton Company, Reno, NV) was used to inject the appropriate targets into the right and/or left hemispheres (FIG. 5D). The injection was performed manually at a rate of 5 μl per minute, a total of either 25 microliters per injection (n of 4, bilateral injections) or 10 microliters (n of 3, unilateral injections), with a subsequent post-injection dwell time of 5 minutes for minimization of reflux. The needle was then withdrawn slightly and a 10 μl air bubble injected to plug the injection well. A skin closure was performed in a standard fashion.

### Magnetic Resonance Imaging (MRI)

MRI imaging was obtained either during survival or at endpoint depending on scanner availability. A clinical Siemens 3T Trio MRI scanner with a 64 Channel head coil was used. Briefly, scans included T1 3D sagittal, T1-fluid attenuated inversion recovery (FLAIR), T2 axial, T2 FLAIR sagittal, Gradient Echo (GRE). After infusion of intravenous gadolinium (0.1 mmol/kg), respective post-contrast T1 weighted sequences were obtained. All scans were processed and evaluated using MicroDicom Viewer (MicroDicom Ltd, Sofia Bulgaria).

### Tissue Processing, H & E, and Immunohistochemistry

Animals (either succumbed) or were euthanized prior to or at approximately 28 days after surgery. Following euthanasia, whole brains were collected for histological analysis. Brains were post-fixed in 4% paraformaldehyde overnight, sliced at 5 mm intervals for paraffin embedding through serial gradients of xylene and ethanol, and sectioned at 8 μm thickness. Hematoxylin and Eosin staining (H & E) was performed in a standard fashion (Hematoxylin Gill No. 3, Sigma Aldrich, Cat: GHS332; Eosin Y, Sigma Aldrich, Cat: HT110132). Immunohistochemistry (IHC) was performed with primary antibodies for GFAP (Dako, Z0034), Vimentin (abcam, ab45939), Ki-67 (abcam, ab15580), EMA (abcam, ab 15481), Progesterone Receptor (PR) (abcam, ab16661), PDGFRA (abcam, ab203491), Olig2 (abcam, ab109186), NG2 (abcam, ab129051), SOX2 (abcam, ab92494), and Neurofilament (NF) (Dako, clone2F11) with subsequent diaminobenzidine (DAB) staining (Vector Laboratories, PK8200). Whole slides were scanned in a raster pattern at 40× magnification (Leica Aperio AT2 Slide Scanner) with calibrated scale bars prepared in ImageJ. Qualitatively, H & E sections were evaluated by two board-certified clinical neuropathologists. Immunohistochemical stains were qualitatively evaluated (neurofilament, GFAP, Vimentin, Ki67, SOX2, Olig2, NG2, PDGFRA).

### Analysis and Statistics

Behavioral and radiographic features are presented with descriptive statistics summarized with absolute and relative frequencies. Continuous and ordinal variables were summarized as appropriate using mean, standard deviation (SD), median and range. Statistical comparisons between were conducted using two-way ANOVA, where P<0.05 considered statistically significant (Prism Graphpad 9, San Diego, CA).

### Primary Cell Culture

Fresh tumor biopsies were obtained from oncogenic vector injection sites (Tumor) or control injection sites (Control) at the time of necropsy. Biopsies underwent trituration and culture in BFP culture media (10/ng/mL PDGFAA, 10 ng/mL bFGF, and N2-Supplement) following an adapted Mayer-Proschel protocol.

### Cell Proliferation Assays

The cell survival assay was used with the 3-(4,5-dimethylthiazol-2-yl)-2,5-diphenyltetrazolium bromide (MTT). The primary cells were seeded (1×10+) in the growth medium for 5 days. Every day, 10 μL of MTT (5 mg/mL in PBS, Sigma, USA) was added to each well and the plates were incubated for 4 h at 37° C. The formazan product was dissolved with 100 μL DMSO. The absorbance at a wavelength of 490 nm was recorded using a microplate reader (BioTek Instruments Inc., USA).

### Immunofluorescence Staining

The primary cells were then fixed in 4% formaldehyde and incubated with primary antibodies, overnight at 4° C. Then, the cells were also incubated with specific secondary antibody for 2 h at room temperature. The nuclei were counterstained with DAPI. The figures were taken by microscopy. All tissue sections were stained with Opal 7-Color Kits (Akoya Biosciences, Menlo Park, CA). The following primary antibodies were used: H-Ras (Santa Cruz, sc-29), PDGFB (Santa Cruz, sc-365805), p53 (CST, #2524), C/EBPβ(Santa Cruz, #7962), NQO1 (CST, #3187), GSTP1 (CST, #3369), Desmin (Invitrogen, MA106401), CD31 (BioRad, MCA1746GA), Collagen IV (abcam, ab6586), GFP (abcam, ab6556), mCherry (abcam, ab 125096), GFAP (abcam, ab7260), Olig2 (Invitrogen, P21954), PDGFRA (abcam, ab203491), CD68 (BioRad, MCA2317GA). The akoya's Vectra Polaris Imaging System was used to image the slides (Akoya Biosciences, Menlo Park, CA).

### Discussion

The present study illustrates the first successful approach to lentiviral vector induced large mammalian model of intracranial HGG. It was found that HGG formed in a highly penetrant fashion with MRI and histopathologic confirmation. From a methodological standpoint, it appears that the volume of injection is critical to the feasibility of this approach. The study also demonstrates that stereotactic lentiviral vector injections inducing the expression of PDGF-B, HRAS, and shP53 induces the formation of HGG in a highly penetrant fashion. The surgical techniques employed to develop this approach using intraoperative neuronavigation appear to be successful, where 8/8 targets were successfully injected and with 8/8 targets forming tumors within the allotted duration of the pilot study using 25 μL of vector. In contrast, lower volume injections created masses in ⅔ animals. While the concept of utilizing neuronavigation for targeting CNS structures in pigs is not new techniques employing this are few given the need for neurosurgical expertise and none have employed this for the purpose of vector driven glioma modeling.

At 50 μL injections, pigs experienced neurological deterioration leading to early termination. However, at 25 μL of injection, pigs continued to be asymptomatic by the pre-determined 4-week endpoint. This is consistent with the prior studies on volume of injections in the central nervous system.

The tumors generated in our study were immunopositive for well-known glioma markers GFAP, Olig2, NG2, SOX2, and PDGFRA. Furthermore, all tumors were found to have a high proliferative index on Ki-67 staining with a mean of 44.1% (SD: +13.6). In addition, the tumors were found to be vimentin positive as well. This is consistent with the previous work in the porcine spinal cord where the generation of HGG is demonstrated.

Additionally, in the present study, regions of inflammatory infiltrate are appreciated in the leptomeningeal spaces and in the tumor bulk illustrating an inflammatory process in the tumors either in-response to the vector, suppressing tumor growth or promoting tumor growth. This would be consistent with a mesenchymal subtype GBM whereby as much as 50% of the tumor bulk can be comprised of inflammatory cells. The vector injections due to the surgical approach have induced glioma formation but have also caused a parallel inflammatory process to occur. This is due to the larger leptomeningeal interface in the brain, and the unavoidable issue of vector reflux into the leptomeninges. This is supported by the fact that the previous work in the spinal cord, whereby there is a significantly more restrained leptomeningeal space and a direct pathway to the lateral white matter, does not illustrate a large inflammatory response in 6/6 animals using identical vectors. A possible solution to this is targeting a deeper white matter structure such as the forceps minor of the corpus callosum or direct callosal injection through an interhemispheric surgical approach.

Given that these tumors were in part generated by upregulating the RTK/RAS/PI3K which is known to induce high levels of oxidative stress, it was examined whether or not anti-oxidative processes were upregulated in these tumors. It is identified, that anti-oxidative candidates known to be upregulated in human GBM, including NADPH quinone oxidoreductase 1 (NQO1), and Glutathione-S-transferase Pi 1 (GSTP1), as well as the enhancer C/EBPβ as responsible for mediating anti-oxidative in glioma cell lines. Marked immunopositivity was found in the three genes in the porcine tumors. While the aforementioned studies focused on EGFRvIII derived cell lines, given the upregulation of these anti-oxidative species, they may be candidates for putative pre-clinical study using novel agents identified targeting redox pathways. Extensive and irregular endothelial cell/pericyte margins and basement membrane structure were also observed consistent with what is found in GBM on Desmin, CD31, and Type IV Collagen staining compared to control tissue. Overall, from a radiologic, histopathologic, and immunohistochemical standpoint, these findings represent a major step for modeling HGG in the minipig brain.

Lastly, the data demonstrates the successful culturing of porcine tumors with relevant immunofluorescent characterization, illustrating a powerful potential tool for future study. One strategy that may be adopted is simply re-transplantation as an allograft model. Given that the selected breed, Göttingen Minipigs, are in-bred as compared to other breeds and demonstrate relatively consistent genetic makeup, it stands to reason that glioma neurospheres may be a next step toward this modeling agenda in large animals. In addition, while the work is examining the formation of alternate tumors in the brain and spinal cord using varied transgenes, tissue culture models from the pig could be employed as a high-throughput screening methodology as an alternative higher-order species in a more clinically relevant space.

Despite significant advances in the knowledge of GBM, there have been limited improvements in outcomes. In part, this represents the recalcitrance of GBM. On the other hand, this highlights the limitations of surgical translation based on existing rodent models, which has stifled the translation of neurosurgical strategies. The study challenges the notion of pre-clinical translational reliance on phenotypically normal large mammalian models used by investigators and regulatory bodies in the pathway for developing neurosurgical strategies. This is a major limitation. For example, in prior studies to bring novel drug delivery strategies to clinical trials, groups have been unable to rigorously study drug delivery variables or drug tracking in a large animal disease model. As such, the development of a highly characterized large mammalian model of GBM fills a translational gap in the field for the study pre-clinical neurosurgical strategies. Indeed, the development of numerous technologies including convection enhanced delivery, oncolytic vectors, laser interstitial thermal therapy (LITT), focused ultrasound (FUS), intraoperative surgical guidance (e.g. 5-ALA), intra-arterial delivery, and robotic resection would benefit from a readily available highly characterized immunocompetent large animal model of GBM.

Large animal models for translational oncologic research are an evolving field with numerous examples arising including severe combined immunodeficiency pigs (SCID) with transplantation of xenografts or modified stem cells, as well as genetically modified pigs with inducible expression of TP53R167 or KRASG12D. The rational underlying this rise in popularity is principally due to two reasons for failure of new oncologic therapeutics in clinical trials, namely toxicity or lack of efficacy. This is especially true for neuro-oncologic tumors, particularly GBM, where few therapeutic strategies move beyond Phase I trials, and ultimately patients continue to face an abysmal prognosis. Currently, for modeling GBM, only two groups have used either U87 or G6 cell line based xenografts with concurrent immunosuppression, transplanting them into Landrace farm-pigs or Yucatan minipigs with macroscopic and histopathologic tumor growth. The use of G6 only produced tumor formation in only one of six animals. However, U87 cell line xenografts were more penetrant in tumor formation yielding tumor growth in 27 of 20 animals (93%) across three studies.

Prior study by the group has demonstrated the use of lentiviral vector mediated delivery to induce high grade spinal-cord glioma in Göttingen minipigs with 6/6 (100%) of animals yielding high grade lesions and a mesenchymal transcriptomic subtype. Prior to the present study, this strategy of vector driven GBM modeling in a large animal, and in the intracranial setting, was not attempted. Fundamentally, the study has established the feasibility of modeling supratentorial GBM in a large mammalian model. Current application also emphasizes characterization of growth rate, advanced radiologic features (positron emission tomography, magnetic resonance spectroscopy), the selection of further neuropathologically relevant genetic lesions (e.g. CDKN2A, EGFR, PTEN, IDH) and the transcriptomic and immunologic phenotype of these tumors. By doing so, these systems provide not only advanced anatomic space for pre-clinical investigation of neurosurgical techniques, but also the potential for evaluation of response to therapy and in immunocompetent space.

The current study demonstrates that it is feasible to achieve glioma growth in the minipig brain. In addition, the study helps evaluate the transcriptomic profile or methylation status of these tumors which is critical for investigating the molecular phenotype and/or potential response to therapeutic agents. Lastly, the surgical approach used in the present study provides a practical means to reach the subcortical white matter in a consistent fashion. It is observed that deeper white matter structures, such as the corpus callosum, can be used for model optimization and use neuronavigation for precise targeted delivery.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will appreciate that numerous changes and modifications can be made to the preferred examples of the invention and that such changes and modifications can be made without departing from the spirit of the invention. It is, therefore, intended that the appended claims cover all such equivalent variations as fall within the true spirit and scope of the invention.

### REFERENCES

Assanah, M., et al., Glial progenitors in adult white matter are driven to form malignant gliomas by platelet-derived growth factor-expressing retroviruses. J Neurosci, 2006. 26 (25): p. 6781-90.
Bedard, P. L., et al., Tumour heterogeneity in the clinic. Nature, 2013. 501 (7467): p. 355-64.
Cancer Genome Atlas Research, N., Comprehensive genomic characterization defines human glioblastoma genes and core pathways. Nature, 2008. 455 (7216): p. 1061-8.
Chen, L., et al., Vertebrate animal models of glioma: understanding the mechanisms and developing new therapies. Biochim Biophys Acta, 2013. 1836 (1): p. 158-65.
D'Amico, R. S., et al., Validation of an effective implantable pump-infusion system for chronic convection-enhanced delivery of intracerebral topotecan in a large animal model. J Neurosurg, 2020. 133 (3): p. 614-623.
Ellis, J. A., et al., Retroviral delivery of platelet-derived growth factor to spinal cord progenitor cells drives the formation of intramedullary gliomas. Neurosurgery, 2012. 70 (1): p. 198-204; discussion 204.
Felix, B., et al., Stereotaxic atlas of the pig brain. Brain Res Bull, 1999. 49 (1-2): p. 1-137.
Gutierrez, J., et al., Preclinical Validation of Multilevel Intraparenchymal Stem Cell Therapy in the Porcine Spinal Cord. Neurosurgery, 2015. 77 (4): p. 604-12; discussion 612.
Hicks, W. H., et al., Large Animal Models of Glioma: Current Status and Future Prospects. Anticancer Res, 2021. 41 (11): p. 5343-5353.
Jacobs, V. L., et al., Current review of in vivo GBM rodent models: emphasis on the CNS-1 tumour model. ASN Neuro, 2011. 3 (3): p. e00063.
Kantor, B., et al., Methods for gene transfer to the central nervous system. Adv Genet, 2014. 87: p. 125-97.
Lenting, K., et al., Glioma: experimental models and reality. Acta Neuropathol, 2017. 133 (2): p. 263-282.
Lynes, J., et al., Lentiviral-induced high-grade gliomas in rats: the effects of PDGFB, HRAS-G12V, AKT, and IDH1-R132H. Neurotherapeutics, 2014. 11 (3): p. 623-35.
Malbert, C. H., Open-source 3D printable frameless stereotaxic system for young and adult pigs. J Neurosci Methods, 2021. 359: p. 109222.
Massey, S. C., et al., Simulating PDGF-Driven Glioma Growth and Invasion in an Anatomically Accurate Brain Domain. Bull Math Biol, 2018. 80 (5): p. 1292-1309.
Merkl, C., et al., RNA interference in pigs: comparison of RNAi test systems and expression vectors. Mol Biotechnol, 2011. 48 (1): p. 38-48.
Nagarajan, P. P., et al., Lentiviral-Induced Spinal Cord Gliomas in Rat Model. Int J Mol Sci, 2021. 22 (23).
Patel, A. P., et al., Single-cell RNA-seq highlights intratumoral heterogeneity in primary glioblastoma. Science, 2014. 344 (6190): p. 1396-401.
Rahme, G. J., et al., A recombinant lentiviral PDGF-driven mouse model of proneural glioblastoma. Neuro Oncol, 2018. 20 (3): p. 332-342.
Sauleau, P., et al., The pig model in brain imaging and neurosurgery. Animal, 2009. 3 (8): p. 1138-51.
Sonabend, A. M., et al., Prolonged intracerebral convection-enhanced delivery of topotecan with a subcutaneously implantable infusion pump. Neuro Oncol, 2011. 13 (8): p. 886-93.
Weber-Levine, C., et al., Porcine Model of Spinal Cord Injury: A Systematic Review. Neurotrauma Rep, 2022. 3 (1): p. 352-368.
Wei, K. C., et al., Neuronavigation-guided focused ultrasound-induced blood-brain barrier opening: a preliminary study in swine. AJNR Am J Neuroradiol, 2013. 34 (1): p. 115-20.

1. A large non-human mammal model of intracranial cancer, comprising:
an intracranial cancer engineered large non-human mammal with a combination of viral vectors encoding oncogenes and shRNA targeting tumor suppressor genes; and
a population of intracranial cancer cells from the large non-human mammal model.

2. The large non-human mammal model of claim 1, wherein the shRNA targeting the tumor suppressor genes comprises sh787 or sh944, wherein the shRNA are operably linked to an H1 promoter or a U6 promoter.

3. (canceled)

4. The large non-human mammal model of claim 1, wherein the tumor suppressor genes comprise CDKN2A, PTEN or p53.

5. The large non-human mammal model of claim 1, wherein the oncogenes comprise platelet-derived growth factor receptor alpha (PDGFRA), platelet-derived growth factor beta (PDGFRB), mutant or wildtype isocitrate dehydrogenase (IDH), mutant or wild-type epidermal growth factor receptor (EGFR), mutant histone H3.3 or mutated Harvey rat sarcoma viral oncogene (HRAS-G12V).

6. The large non-human mammal model of claim 5, wherein the PDGFRA, PDGFRB, IDH, EGFR, mutant histone H3.3, or HRAS-G12V is operably linked to an Ef1α promoter.

7. The large non-human mammal model of claim 1, wherein the combination of viral vectors comprises a recombinant adenoviral vector, a recombinant adeno-associated viral vector (AAV), a herpes simplex virus type 1 vector (HSV), a moloney murine leukemia virus (MMLV) vector or a lentiviral vector.

8. The large non-human mammal model of claim 1, wherein each viral vector in the combination of viral vectors is encapsulated in a nanoparticle, a polymer, or a liposome.

9. The large non-human mammal model of claim 1, wherein the large non-human mammal is a Gottingen minipig.

10. The large non-human mammal model of claim 1, wherein the intracranial cancer comprises brain metastasis, high-grade glioma (HGG), low-grade glioma (LGG), meningioma, cerebral arteriovenous malformation, vestibular schwannoma, pituitary adenoma, neuroblastoma, or gliosarcoma.

11. (canceled)

12. A method of growing an intracranial tumor in a large non-human mammal model, comprising:
administering a combination of viral vectors encoding oncogenes and shRNA targeting tumor suppressor genes at an intracranial delivery location in the large non-human mammal, thereby inducing intracranial tumor;
obtaining a biopsy sample from the intracranial tumor; and
culturing cells from the biopsy sample.

13. (canceled)

14. (canceled)

15. (canceled)

16. The method of claim 12, wherein the combination of viral vectors comprises each viral vector titer at about 10⁸ to about 10⁹ infectious units (IU)/ml.

17. The method of claim 12, wherein the shRNA targeting the tumor suppressor genes comprises sh787 or sh944, wherein the shRNA are operably linked to an H1 promoter or a U6 promoter.

18. The method of claim 12, wherein the tumor suppressor genes comprise cyclin-dependent kinase inhibitor 2A (CDKN2A), PTEN or p53.

19. The method of claim 12, wherein the oncogenes comprise platelet-derived growth factor receptor alpha (PDGFRA), platelet-derived growth factor beta (PDGFRB), mutant or wildtype isocitrate dehydrogenase (IDH), mutant or wildtype epidermal growth factor receptor (EGFR), mutant histone H3.3 or mutated Harvey rat sarcoma viral oncogene (HRAS-G12V).

20. The method of claim 19, wherein the PDGFRA, PDGFRB, IDH, EGFR, mutant histone H3.3, or HRAS-G12V is operably linked to an Ef1α promoter.

21. The method of claim 12, wherein the combination of viral vectors comprises a recombinant adenoviral vector, a recombinant adeno-associated viral vector (AAV), a herpes simplex virus type 1 vector (HSV), a moloney murine leukemia virus (MMLV) vector, or a lentiviral vector.

22. (canceled)

23. The method of claim 12, wherein the large non-human mammal is a Gottingen minipig.

24. The method of claim 12, wherein the intracranial tumor comprises brain metastasis, glioblastoma, high-grade glioma (HGG), low-grade glioma (LGG), meningioma, cerebral arteriovenous malformation, vestibular schwannoma, pituitary adenoma, neuroblastoma, or gliosarcoma.

25. (canceled)

26. The method of claim 12, wherein the combination of viral vectors encoding oncogenes and shRNA targeting tumor suppressor genes is delivered concurrently or sequentially.

27.-37. (canceled)

38. A combination of recombinant viral vectors to induce brain disorders in a large non-human mammal model, comprising:
a gene, wherein the gene consists of epidermal growth factor receptor (EGFR), platelet-derived growth factor B (PDGFB), Harvey rat sarcoma viral oncogene homolog (HRAS), epidermal growth factor receptor (EGFRvIII), B-Raf proto-oncogene, serine/threonine kinase mutation at valine 600 to glutamic acid (BRAF V600E), histone H3 mutation at lysine 27 to methionine (H3K27M), wild-type isocitrate dehydrogenase 1 (IDH1WT), isocitrate dehydrogenase 1 mutation at arginine 132 to histidine (IDH1 R132H), and isocitrate dehydrogenase 1 (IDH1); and
a short hairpin RNA targeting tumor suppressor genes, wherein the tumor suppressor genes consist of phosphatase and tensin homolog (PTEN), cyclin-dependent kinase inhibitor 2A (CDKN2A), and protein TP53 (p53).
